(19) United States
(12) Patent Application Publication
(10) Pub. No.: US 2002/0045173 A1
(43) Pub. Date: Apr. 18, 2002

(54) GLP-2 receptor gene promoter and uses thereof

(76) Inventors: Daniel J. Drucker, Toronto (CA); Julie Lovshin, Toronto (CA)

Correspondence Address: Stephen A. Bent, FOLEY & LARDNER, Washington Harbour, 3000 K Street, N.W., Suite 500, Washington, DC, 20007-5109, US

(21) Appl. No.: 09/833,740

(22) Filed: Apr. 13, 2001

Related U.S. Application Data
(60) Provisional application No. 60/196,909, filed on Apr. 13, 2000.
(60) Provisional application No. 60/265,310, filed on Feb. 1, 2001.

(51) Int. Cl. C12Q 1/68; A01K 67/00; C07H 21/04; C07K 14/705; C12P 21/02; C12N 5/06

(52) U.S. Cl. 435/6; 800/8; 435/325; 435/320.1; 530/350; 536/23.5; 435/69.1

(57) ABSTRACT

The invention is directed to the promoter region of the GLP-2 receptor gene and uses thereof.

This application claims priority from U.S. provisional application Ser. No. 60/196,909 filed Apr. 13, 2000 and Ser. No. 60/265,310 filed Feb. 1, 2001.

### FIELD OF THE INVENTION

This invention is in the field of molecular biology. More particularly, the invention relates to promoters useful to drive expression of genes of interest in a tissue-specific manner particularly in the gut and brain, to expression cassettes containing such promoters and their use as drug screening tools and as protein delivery vehicles, and to cells and organisms containing such expression cassettes.

### BACKGROUND OF THE INVENTION

Glucagon-like peptide 2 (GLP-2), a 33 amino acid product of the preproglucagon gene, has been described as a potent growth factor for gastrointestinal tissue, including the large bowel, upper GI, and, particularly, the small bowel, which acts by stimulating cellular proliferation and inhibition of cell death. Drucker et al., Proc. Natl. Acad. Sci., 1996, 93:7911. Effects of GLP-2 on satiety suggest that GLP-2 may be useful medically to suppress appetite. The use of GLP-2 to ameliorate damage to gastrointestinal mucosa caused by chemotherapeutics is suggested in WO96/32414, published Oct. 17, 1996. The similar use of GLP-2 in combination with other growth factors such as growth hormone (GH) and insulin-like growth factors (IGF-1 and IGF-2) is also described in WO99/25644, published Jun. 18, 1998.

The glucagon-like peptides are liberated in the gut and central nervous system via tissue-specific posttranslational processing of a common proglucagon precursor. Mojsov et al., J. Biol. Chem., 261:11880-11889 (1986). GLP-1 also promotes expansion of islet mass via stimulation of beta cell proliferation and induction of islet neogenesis via increased ductal pdx-1 expression. Xu et al., (1999) Diabetes 48(12), 2270-6; and Stoffers et al., (2000) Diabetes 49, 741-748.

Glucagon-like peptide-2 (GLP-2) exhibits trophic properties in the small and large bowel characterized by expansion of the mucosal epithelium via stimulation of crypt cell proliferation and inhibition of apoptosis. Drucker et al., (1996) Proc. Natl. Acad. Sci. USA 93, 7911-7916; Tsai et al., (1997) Am. J. Physiol. 272, G662-G668; and Tsai et al., (1997) Am. J. Physiol. 273, E77-E84. GLP-2 also regulates gastric motility and gastric acid release, and intestinal permeability and intestinal hexose transport, actions which are independent of its effects on epithelial growth. Wojdemann et al., (1998) Scand. J. Gastroenterol. 33, 828-832; Wojdemann et al., (1999) J. Clin. Endocrinol. Metab. 84(7), 2513-7; Benjamin et al., (2000) Gut 47, 112-119; and Cheeseman et al., (1996) Am. J. Physiol. Gastrointest. Liver Physiol. 271, G477-G482.

The intestinotrophic and cytoprotective properties of GLP-2 have been evaluated in the setting of acute intestinal injury, where GLP-2 administration inhibits apoptosis and reduces the severity of mucosal damage in both the small and large intestine. Drucker et al., (1999) Am. J. Physiol. 276, G79-G91; Boushey et al., (1999) Am. J. Physiol. 277, E937-E947; Alavi et al., (2000) J. Pediatr. Surg. 35(6), 847-51; and Prasad et al., (2000) J. Pediatr. Surg. 35(2), 357-9.

In the central nervous system, the glucagon-like peptides are synthesized predominantly in the caudal brainstem and to a lesser extent, in the hypothalamus. Drucker et al., (1988) J. Biol. Chem. 263, 13475-13478; Han et al., (1986) J. Neurosi. Res. 16, 97-107; and Larsen et al., (1997) Neuroscience 77, 257-270. The GLP-1 receptor is expressed more widely throughout the CNS (Merchenthaler et al., (1999) J. Comp. Neurol. 403(2), 261-80; and Campos et al., (1994) Endocrinology 134, 2156-2164), and GLP-1 has been shown to regulate appetite, hypothalamic pituitary function, and the central response to aversive stimulation. Turton et al., (1996) Nature 379, 69-72; Beak et al., (1998) J. Clin. Invest. 101, 1334-1341; Beak et al., (1996) Endocrinology 137, 4130-4138; Seeley et al., (2000) J. Neurosci. 20(4), 1616-21; Seeley et al., (2000) Endocrinology 141(2), 473-475; and Rinaman, L. (1999) Am. J. Physiol. 277, R582-90.

In contrast to the increasing number of studies describing CNS actions of GLP-1, much less is known about the potential function(s) of GLP-2 in the brain. Experiments using rat hypothalamic and pituitary membranes demonstrated GLP-2-mediated activation of adenylate cyclase. Hoosein et al., (1984) FEBS Lett. 178, 83-86. Consistent with these findings, the actions of GLP-2 were subsequently shown to be transduced in a cAMP-dependent manner via a recently cloned GLP-2 receptor (GLP-2R) isolated from hypothalamic and intestinal cDNA libraries. Munroe et al., (1999) Proc. Natl. Acad. Sci. USA 96, 1569-1573.

The GLP-2R is expressed in a highly tissue-specific manner predominantly in gut endocrine cells and in the brain. In comparison with GLP-1, little is known about either the expression or function of the GLP-2R in different regions of the CNS. Munroe et al., (1 999) Proc. Natl. Acad. Sci. USA 96, 1569-1573; and Yusta et al., (2000) Gastroenterology 119(3), 744-755.

The cellular targets and molecular mechanisms underlying GLP-2 action remain poorly defined. Munroe et al. report cloning of the GLP-2 receptor from both rat and human cDNA libraries. See PNAS, USA, 1999, 96:1569. The human GLP-2 receptor was mapped to chromosome 17p13.3. Results indicate that the receptor is triggered selectively by GLP-2 relative to either GLP-1 or glucagon. Studies of receptor mRNA distribution revealed that the receptor is expressed in the hypothalamus and along the intestinal epithelium.

### SUMMARY OF THE INVENTION

The transcriptional control region of the GLP-2 receptor gene now has been cloned and utilized to drive expression of a heterologous gene of interest. The expression occurred tissue-selectively, in the epithelium of the intestine, and particularly of the jejunum, ileum and colon, and in the lateral hypothalamus, including the amygdalary-hippocampal nuclei region of the brain.

The present invention thus provides, in one of its aspects, a recombinant DNA expression construct in which a heterologous gene of interest is linked operably with (i.e., under the expression control of) the promoter region of the GLP-2 receptor gene.

In embodiments of the present invention, the gene of interest expressed from the GLP-2 receptor gene promoter region is a reporter gene, permitting detection of GLP-2 receptor promoter function. In another aspect of the present invention, expression constructs of this type are utilized to screen for agents that modulate function of the GLP-2 receptor gene promoter region. For this purpose, the present invention further provides host cells and/or organisms, including animal hosts, that incorporate such expression cassettes and are used to screen chemical compounds to identify agents that modulate GLP-2 receptor gene promoter function, using altered reporter gene expression levels, or altered reporter gene product levels, as a read-out to identify promoter modulators. Such modulators can be used therapeutically to modulate endogenous levels of GLP-2 receptor and thereby influence the endogenous effects of GLP-2, for purposes of regulating its intestinotrophic activity or of regulating the effect of GLP-2 on appetite and satiety.

In a related aspect of the present invention, such recombinant DNA expression constructs are used to identify cells and cell lines in which the GLP-2 receptor gene promoter region is functional, including but not limited to those cells and lines that endogenously express the GLP-2 receptor. Particularly, candidate cells are transfected with the expression construct incorporating the reporter gene, and expression of the reporter gene reveals those cells in which the GLP-2 receptor gene promoter region is functional.

In another embodiment of the present invention, the gene of interest expressed from the GLP-2 receptor gene promoter region is a gene coding for a therapeutic protein product. Such expression cassettes can be utilized to introduce selected gene products into the gut and brain tissues in which the endogenous GLP-2 receptor gene normally functions.

Thus, in one aspect of the present invention, a method is provided for delivering a gene product, to tissues selected from the intestinal epithelium and the lateral hypothalamus, by transfecting an organism, or a gametic or embryonic form of such an organism, with an expression construct in which the selected gene is linked for expression to the GLP-2 receptor gene promoter region. A wide variety of medically useful gene products can be delivered in this manner. The present invention thus further provides transgenic cells and/or organisms, including non-human animals, that incorporate such expression cassettes and which therefore produce a gene product of interest under the control of the GLP-2 receptor gene promoter region.

These and other aspects of the present invention are now described with reference to the accompanying drawings:

### BRIEF DESCRIPTION OF THE DRAWINGS

FIG. 1 provides the nucleotide sequence of a 5′-flanking and 5′-untranslated region (UTR) of a GLP-2 receptor gene.

FIG. 2 provides a comparison of the sequence encoding the putative 5′-UTR sequence in the murine gene to the 5′UTR sequence of the rat cDNA.

FIG. 3 illustrates the strategy employed to amplify the 5′UTR of rat GLP-2 cDNA and identify a counterpart murine UTR.

FIG. 4 shows a schematic map of the promoter region of the murine GLP-2 receptor gene, predicted by Southern blot analysis.

FIG. 5 shows RT-PCR detection of transgene expression from a GLP-2 receptor gene promoter region in gastrointestinal tissue of transgenic mice.

FIG. 6 shows β-galactosidase activity in the cortical amygdala and hippocampus of the transgenic mice.

FIG. 7a shows approximately 230 bp of sequence, including 104 bp of 5′-untranslated sequences corresponding to the 5′-end of the cDNA encoding the rat GLP-2R obtained from sequencing of RACE products.

FIG. 7b shows the organization of 5′-flanking and exon-1 sequences in the mouse GLP-2R gene compared to rat exon 1 and human GLP-2R 5′-flanking and 5′-untranslated sequences.

FIG. 7c shows construction of the transgene achieved by inserting a 1.5-kb Sma I-Pst I fragment of the murine GLP-2R gene upstream of an nlsLacZ cDNA.

FIG. 8a shows the RT-PCR analysis of transgene (nlsLacZ) expression in two different lines of 1.5-kb GLP-2R promoter-LacZ transgenic mice and endogenous GLP-2R mRNA transcripts in gastrointestinal tissues.

FIG. 8b shows the RT-PCR analysis of endogenous GLP-2R, 1.5 kb GLP-2R promoter-LacZ (nlsLacZ), and GAPDH mRNA transcripts in structures isolated from the CNS of GLP-2R promoter-LacZ transgenic mice.

FIG. 8c shows the analysis of the tissue-specificity of transgene expression in liver, kidney, lung, spleen, and heart from 1.5-kb GLP-2R promoter-LacZ transgenic mice.

FIG. 8d shows the analysis of transgene expression in frozen histological sections (kidney and brain) from 1.5-kb GLP-2R promoter-LacZ transgenic mice and littermate control mice incubated with β-galactosidase antiserum.

FIG. 9a shows a slide of frozen coronal sections of formulin-perfused rat cerebellum exposed to GLP-2R antiserum.

FIG. 9b shows a slide of frozen coronal sections of formulin-perfused rat cerebellum exposed to preimmune serum.

FIG. 9c shows a slide of frozen coronal sections of formulin-perfused mouse cerebellum exposed to GLP-2R antiserum.

FIG. 9d shows a slide of frozen coronal sections of formulin-perfused mouse cerebellum exposed to preimmune serum.

FIG. 9e shows a slide of frozen coronal sections of formulin-perfused GLP-2R promoter-LacZ mouse cerebellum incubated with a polyclonal antibody generated against the β-galactosidase enzyme to detect specific nuclear LacZ expression in the cerebellum of GLP-2R promoter-LacZ mice.

FIG. 9f shows a slide of frozen coronal sections of formulin-perfused littermate control mouse cerebellum incubated with a polyclonal antibody generated against the β-galactosidase enzyme to detect specific nuclear LacZ expression in the transgenic cerebellum.

FIGS. 10a-c show slides of frozen coronal sections of perfused rat brain incubated with GLP-2R antiserum to detect the presence of GLP-2R positive cell types in the hippocampus and dentate gyrus. FIG. 10a shows endogenous GLP-2R immunostaining in rostral hippocampus,

FIG. 10b shows staining in the granular layer of dentate gyrus, and

FIG. 10c shows staining in the CA3 field of hippocampus.

FIGS. 10d-h shows slides of frozen coronal sections of perfused GLP-2R promoter-LacZ mouse brain incubated with β-galactosidase antibody to identify transgene positive nuclei in the transgenic hippocampus and dentate gyrus. The boxed regions of FIG. 10d correspond to areas containing LacZ+ positive cells including the transgenic dentate gyrus (shown in FIGS. 10e and g) and the CA1 (shown in FIG. 10f) field of the transgenic hippocampus using β-galactosidase immunocytochemistry. FIG. 10h shows a slide of the caudal CA3 field of the transgenic hippocampus using X-GAL histochemical analysis.

FIG. 11a shows a slide of a histochemical analysis of LacZ positivity in the amygdaloid nucleus of GLP-2R promoter-LacZ transgenic mice using X-GAL staining. The boxed area corresponds to LacZ+ nuclei in the amygdalohippocampal area and the arrows point to LacZ+ nuclei in the caudal CA3 region of the hippocampus and the posteromedial cortical amygdaloid nucleus.

FIGS. 11b and c show a slide of an immunocytochemical analysis of the amygdalohippocampal area (FIG. 11b) and the posteromedial (FIG. 11c) cortical amygdaloid nucleus using an antibody directed against the p-galactosidase enzyme.

FIGS. 11d-f show a slide of an immunocytochemical analysis of the hypothalamus of GLP-2R promoter-LacZ transgenic mice using β-galactosidase antiserum, specifically in the dorsomedial nucleus (FIG. 11d) and in the ventromedial nucleus (FIG. 11e) 60× and (FIG. 11f) 240× magnification.

FIGS. 11g-i show a slide of an immunocytochemical analysis of thalamic nuclei in GLP-2R promoter-LacZ transgenic mice using β-galactosidase antiserum, specifically in the mediodorsal nuclei (FIG. 11g) the dorsomedial part of the laterodorsal thalamic nucleus (FIG. 11h) and the ventrolateral geniculate nucleus (FIG. 11i). The arrows point to LacZ+ nuclei.

### DETAILED DESCRIPTION OF THE PREFERRED EMBODIMENTS

The present invention exploits the promoter region of the GLP-2 receptor gene. The GLP-2 receptor gene (“GLP-2R gene”) is characterized as that region of genomic DNA that mediates production of a GLP-2 receptor having the structural and functional properties reported in the literature, for instance, by Munroe et al., supra, incorporated herein by reference.

In other words, the GLP-2R gene includes not only the coding region and the 5′-UTR previously disclosed by Munroe et al. but also a 5′-flanking region, described here, which functions as a tissue-specific promoter in vivo. More specifically, it has been discovered that the promoter region which drives expression of the endogenous GLP-2R gene coding region begins 5′ to the first base of the 5′-UTR and extends upstream therefrom to include, minimally, the number of bases necessary to drive transcription at levels above detectable background. Thus, the promoter region comprises at least about 1,000 bases upstream of the transcription start site, suitably at least about 1,200 bases upstream thereof, and desirably at least about 1,400 bases 5′ thereof. The maximum size of the promoter region can extend beyond about 8,000 bases upstream of the transcription start site, and desirably incorporates those components, such as transcriptional factor binding sites and upstream activator sequences, through which expression from the endogenous GLP-2R gene normally is regulated.

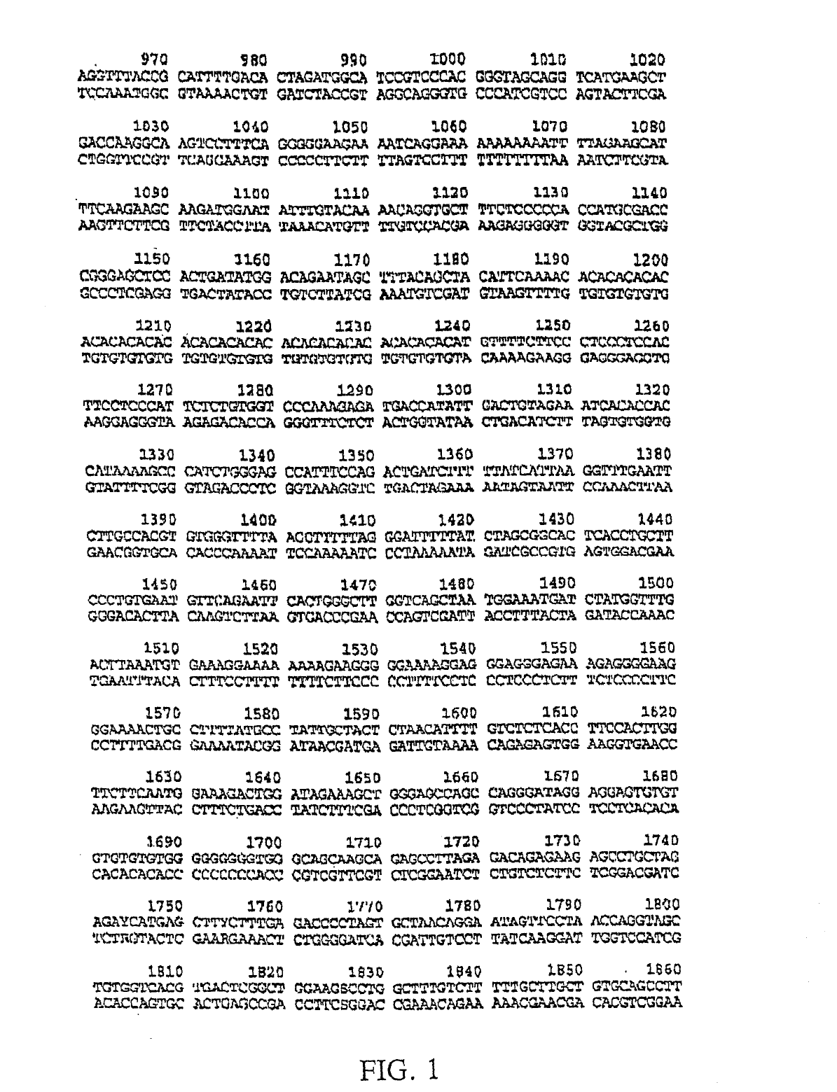

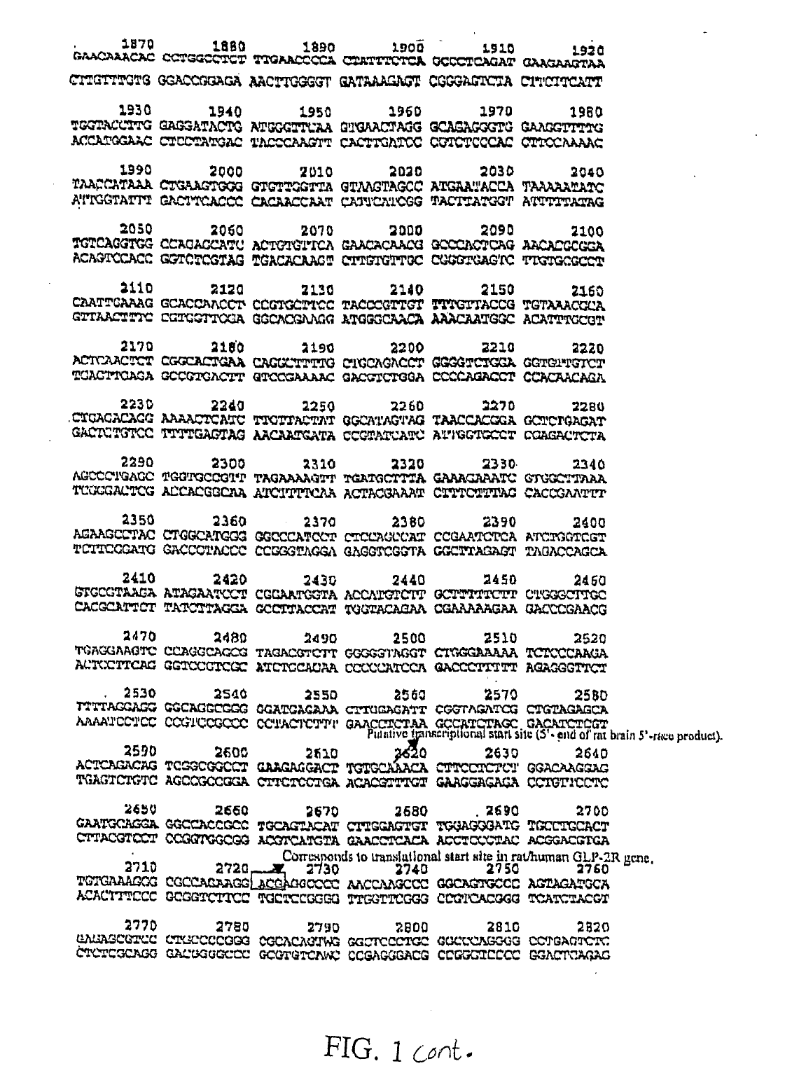

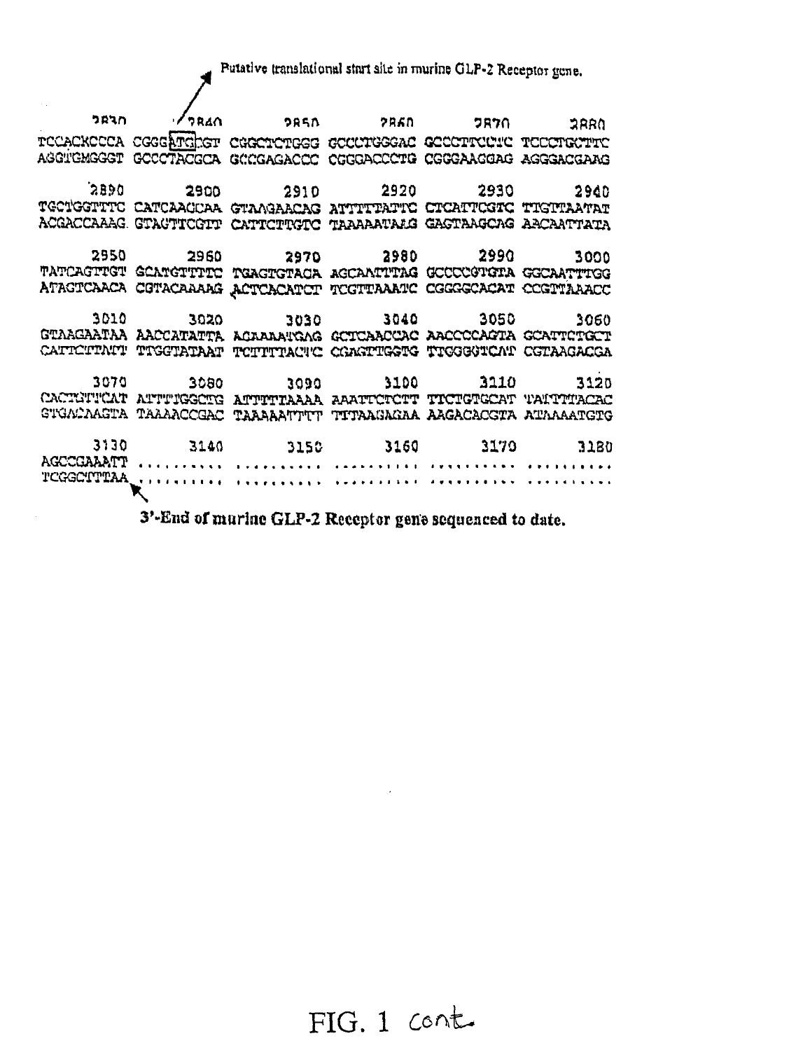

In one embodiment of the invention, the GLP-2R promoter region is constituted by the promoter region of the mouse GLP-2R gene. In specific embodiments of the invention, this promoter region comprises a region of about 1.5 kb upstream of the transcription start site of the mouse GLP-2 coding region, as illustrated in FIG. 1. This regulatory region of the mouse GLP-2R gene was able to drive expression of a reporter gene in transgenic mice, in the tissue-specific manner postulated for the wild type, endogenous GLP-2R gene. The mouse GLP-2R gene promoter region can further comprise additional sequences upstream of the noted 1.5 kb region and can include 2 kb or more from the transcription start site, for instance 3 kb or more. Expression results paralleling those generated using the 1.5 kb promoter region have been achieved also in transgenic mice using a promoter region constituted by about 8 kb upstream of the transcription start site.

It will be appreciated that the promoter region of GLP-2R genes native to other species can also be exploited in the present invention. For instance, promoter regions can also be obtained from the GLP-2R genes native to other vertebrate species including livestock and poultry, and mammals including rodents, pets, primates and humans. In a particular embodiment, the GLP-2R promoter region is constituted by the promoter region associated with the GLP-2R gene endogenous to human cells, particularly to human cells known to express an endogenous GLP-2R gene, such as epithelial cells of the jejunum, the ileum, the colon, the stomach and the amygdalary-hippocampal nuclei.

One skilled in the art will recognize that modest changes to the composition of the promoter region will not disrupt its regulatory function. Since transcription regulation is limited to a few, discrete sequences within the regulatory region, base changes in non-critical sequences will produce minimal changes in gene expression. Thus, the GLP-2R promoter of the present invention includes the promoter region of the mouse GLP-2 receptor gene, homologs thereof including the promoter region of the human GLP-2 receptor gene, as well as variants of the mouse and homologous GLP-2R gene promoter regions that may include truncations, extensions, insertions and deletions within the promoter region, but which retain GLP-2R promoter function as determined by any of the assays herein described.

The GLP-2R gene promoter region can be isolated from genomic DNA or BAC libraries using techniques that are now well established in the art. In cases where the genomic DNA insert incorporates the GLP-2R gene coding region, the insert can be identified by hybridization with labeled cDNAs encoding the GLP-2R protein. Such probes have the coding sequence, for instance, of the human or rat receptor-encoding cDNAs reported by Munroe et al., supra (see also GenBank database Accession Nos. AF105367 and AF105368). Once so identified, the genomic DNA inserts can be manipulated and tailored to excise the promoter region contained therein.

In the alternative, the genomic DNA or BAC library can be screened using, as probe, a sequence constituting a distinguishing portion or all of the mouse GLP-2R promoter regions illustrated in FIG. 1. Hybridizing inserts can then be manipulated to release the promoter region, and then tailored to allow its subsequent use as described below. Similarly, a selected genomic library can be tapped using a PCR technique, such as RT-PCR, with the aid of primers designed on the basis of the mouse GLP-2R gene promoter region depicted in FIG. 1.

Functional variants, for example fragments, analogs or derivatives, can be identified by their ability to hybridize to the complement DNA sequence of the disclosed promoter region under stringent conditions. Suitable hybridization conditions are discussed below.

“Hybridization” is used here to denote the pairing of complementary nucleotide sequences to produce a DNA-DNA hybrid or a DNA-RNA hybrid. Complementary base sequences are those sequences that are related by the base-pairing rules. In DNA, A pairs with T and C pairs with G. In RNA U pairs with A and C pairs with G. Typically, nucleotide sequences to be compared by means of hybridization are analyzed using dot blotting, slot blotting, Northern or Southern blotting. Southern blotting is used to determine the complementarity of DNA sequences. Northern blotting determines complementarity of DNA and RNA sequences. Dot and Slot blotting can be used to analyze DNA/DNA or DNA/RNA complementarity. These techniques are well known by those of skill in the art. Typical procedures are described in CURRENT PROTOCOLS IN MOLECULAR BIOLOGY, Ausubel et al., eds. (John Wiley & Sons, Inc. 1995) (hereafter “Ausubel”), at pages 2.9.1 through 2.9.20.

A probe is biochemically labeled with a radioactive isotope or tagged in other ways for ease in identification. A probe is used to identify a gene, a gene product or a protein. Thus, a polynucleotide probe can be used to identify complementary nucleotide sequences. An mRNA probe will hybridize with its corresponding DNA gene.

Typically, the following general procedure is used to determine hybridization under stringent conditions. A sample polynucleotide is imobilized on a membrane and a DNA sequence complementary to the disclosed regulatory region is used as a “probe.” Using procedures well-known to those skilled in the art, the ability of the probe to hybridize with the sample polynucleotide sequence can be analyzed. Conversely, a DNA sequence complementary to the disclosed regulatory region can be immobilized and a sample polynucleotide can be used as a probe.

One of skill in the art will recognize that various factors can influence the amount and detectability of the probe bound to the immobilized DNA. The specific activity of the probe must be sufficiently high to permit detection. Typically, a specific activity of at least 108 dpm/ug is necessary to avoid weak or undetectable hybridization signals when using a radioactive hybridization probe. A probe with a specific activity of 108 to 109 dpm/ug can detect approximately 0.5 pg of DNA. It is well-known in the art that sufficient DNA must be immobilized on the membrane to permit detection. It is desirable to have excess immobilized DNA and spotting 10 ug of DNA is generally an acceptable amount that will permit optimum detection in most circumstances. Adding an inert polymer such as 10% (w/v) dextran sulfate (mol. wt. 500,000) or PEG 6000 to the hybridization solution can also increase the sensitivity of the hybridization. Adding these polymers has been known to increase the hybridization signal. See Ausubel, supra, at p 2.10.10.

To achieve meaningful results from hybridization between a first nucleotide sequence immobilized on a membrane and a second nucleotide sequence to be used as a hybridization probe, (1) sufficient probe must bind to the immobilized DNA to produce a detectable signal (sensitivity) and (2) following the washing procedure, the probe must be attached only to those immobilized sequences with the desired degree of complementarity to the probe sequence (specificity). “Stringency,” as used in this specification, means the condition with regard to temperature, ionic strength and the presence of certain organic solvents, under which nucleic acid hybridizations are carried out. The higher the stringency used, the higher degree of complementarity between the probe and the immobilized DNA.

“Stringent conditions” designates those conditions under which only polynucleotides that have a high frequency of complementary base sequences will hybridize with each other. Exemplary stringent conditions are (1) 0.75 M dibasic sodium phosphate/0.5 M monobasic sodium phosphate/1 mM disodium EDTA/1% sarkosyl at about 42° C. for at least about 30 minutes, (2) 6.0M urea/0.4% sodium laurel sulfate/0.1% SS× at about 42° C. for at least about 30 minutes, (3) 0.1×SSC/0.1% SDS at about 68° C. for at least about 20 minutes, (4) 1×SSC/0.1% SDS at about 55° C. for about one hour, (5) 1×SSC/0.1% SDS at about 62° C. for about one hour, (6) 1×SSC/0.1% SDS at about 68° C. for about one hour, (7) 0.2×SSC/0.1% SDS at about 55° C. for about one hour, (8) 0.2×SSC/0.1% SDS at about 62° C. for about one hour, and (9) 0.2×SSC/0.1% SDS at about 68° C. for about one hour. See Ausubel, supra, at pages 2.10.1 through 2.10.16, which are hereby incorporated by reference, and Sambrook et al., MOLECULAR CLONING: A LABORATORY MANUAL (Cold Spring Harbor Press, 1989), at §§1.101-1.104.

While stringent washes are typically carried out at temperatures from about 42° C. to about 68° C., one of skill in the art will appreciate that other temperatures may be suitable for stringent conditions. Maximum hybridization typically occurs at about 20 to about 25° C. below the Tm for DNA-DNA hybrids. It is well known in the art that Tm is the melting temperature, or temperature at which two nucleotide sequences dissociate. Methods for estimating Tm are well known in the art. For example, see Ausubel, supra, at page 2.10.8. Maximum hybridization typically occurs at about 10 to about 15° C. below the Tm for DNA-RNA hybrids.

Functional variants also can be identified by comparing their structural similarity, or homology, to the disclosed regulatory region. A DNA fragment possessing 75% or more sequence homology, especially 85-95%, to the GLP-2R gene promoter region is considered a functional variant and is encompassed by the present invention. Mathematical algorithms, for example the Smith-Waterman algorithm, also can be used to determine sequence homology. See Smith and Waterman, J. Mol. Biol. 147:195-197 (1981); Pearson, Genomics, 11:635-650 (1991). Although any sequence algorithm can be used to identify functional variants, the present invention defines functional variants with reference to the Smith-Waterman algorithm, where the GLP-2R gene promoter region, or a portion thereof, is used as the reference sequence to define the percentage of homolology of polynucleotide homologues over its length. The choice of parameter values for matches, mismatches, and inserts or deletions is arbitrary, although some parameter values have been found to yield more biologically realistic results than others. One preferred set of parameter values for the Smith-Waterman algorithm is set forth in the “maximum similarity segments” approach, which uses values of 1 for a matched residue and −⅓ for a mismatched residue (a residue being a either a single nucleotide or single amino acid). Waterman, Bulletin of Mathematical Biology 46:473-500 (1984). Insertions and deletions x, are weighted as

xk=1+k/3,

where k is the number of residues in a given insert or deletion (Id.).

Preferred variant polynucleotides are those having about 75% sequence homology to the GLP-2R gene promoter region using the Smith-Waterman algorithm. Particularly preferred variant polynucleotides have at least about 90% sequence homology. Even more preferred variant polynucleotides have at least about 95% sequence homology, and most preferred variant polynucleotides have at least 98% sequence homology.

In accordance with aspects of the invention, the GLP-2R gene promoter region (“the GLP-2R promoter”) is utilized in the form of a recombinant DNA expression construct, in which the GLP-2R promoter is linked operably for expression to a gene of interest. In a particular aspect of the present invention, the gene of interest is a gene useful to report functioning of the GLP-2R promoter, and therefore is denoted a “reporter gene.” In another aspect of the present invention, the gene of interest is a gene that either is itself a product of therapeutic value, or codes for an expression product of therapeutic value. These recombinant DNA expression constructs comprise the GLP-2R promoter in operable linkage with the gene of interest, to cause expression of the gene of interest from the GLP-2R promoter when the construct is placed in an expression environment such as a cell or organism. The preparation of recombinant DNA expression constructs is well-known to those of ordinary skill in the art. See Ausubel, supra.

Useful reporter genes are well known in the art, and include genes encoding luciferase, chloramphenicol acetyltransferase, β-glucuronidase, β-galactosidase, neomycin phosphotransferase, or guanine xanthine phosphoribosyltransferase, among many others. A suitable reporter gene also may encode the therapeutic proteins of interest, as noted below, or may be any gene that can be identified with convenience or whose expression product can be identified via a convenient assay, such as an immunoassay, a fluorescence or chemiluminescence assay or a cell survival assay.

The reporter gene constructs of the invention are useful in a variety of ways, including (1) to identify cells capable of mediating expression from the chosen GLP-2R promoter, (2) to identify regions of the GLP-2R promoter that are functional in a given cell type, and (3) to screen for agents that modulate expression from the GLP-2R promoter. In the first mentioned utility, cells or cell lines can be screened as potential hosts for expression from the GLP-2R promoter. Among the most suitably targeted hosts are cells and cell lines of the tissues of the gut and the hippocampal region of the brain, demonstrated by the examples herein as having the ability to support GLP-2R promoter-driven expression. To this end, the reporter gene construct can then be transfected into the selected host cells using standard procedures. Upon culturing of the cells, detection of the reporter gene indicates that the selected cell will support and mediate expression from the GLP-2R promoter. There is thus identified a cell line that can be exploited to drive expression of genes of therapeutic interest, for the purpose of producing that gene product either in vitro for large scale commercial production, or in vivo as part of a gene therapy strategy.

The reporter gene constructs can also be used to map functional regions of the GLP-2R promoter. In this respect, the construct can be manipulated using standard molecular biology techniques to introduce variation in the promoter region, such as by 5′ truncation, point and regional mutation, addition and deletion, and the like, thereby to identify regions of the promoter that are critical to its activity and the regulation of that activity. As will be understood, the effect of such variation on activity can be determined by culturing cells harboring the reporter construct, and determining the effect of the promoter variation on reporter gene expression levels relative to levels of reporter gene expression from a wild type promoter.

The reporter gene constructs are also useful, in accordance with another aspect of the invention, to screen for agents that modulate the function of the GLP-2R promoter. Such agents will find medical use in the treatment of various conditions, disorders and diseases. In the case of agents that upregulate GLP-2 receptor expression, it is anticipated that the use of such agents can increase the intestinal benefits available from GLP-2, by increasing the GLP-2 receptor population available for triggering by the endogenous GLP-2. Such agents thus can be used to elicit increases in intestinal mass and function, to improve gut barrier function, and to control gut motility. In the brain, such agents can enhance the satiety function contributed by GLP-2, leading to increase appetite suppression and a treatment for obesity and related disorders. Agents that down-regulated function of the GLP-2R promoter can find use in the control of intestinal cell proliferation, particularly to treat certain cancers and neoplasms, and to elevate appetite in patients suffering from anorexia and related conditions.

To identify agents that modulate GLP-2R promoter function, the reporter gene constructs, and cells containing them, can be exploited in a high throughput screening protocol, whereby cultured host cells are incubated with candidate modulators, and the effect of the candidate modulator on reporter gene expression levels is determined relative to control cells. Candidates that cause a difference in reporter gene expression levels are identified as modulators using routine techniques, as described particularly in U.S. Pat. No. 5,665,543 to Oncogene Science, incorporated herein by reference.

The candidate modulating agents can be identified within vast chemical libraries, using a random screening approach. Particularly suitable agents for screening are those agents known to have an effect on the binding, to the GLP-2R promoter, of transcriptional factors that influence the function of that promoter. The identification of these transcription factors, and the sequence motif within the promoter region to which they bind, can be accomplished using established algorithms. One such program useful to identify these motifs and corresponding transcription factors is known as TFSEARCH, and is available on line at http://pdap 1.trc.rwap.or.jp/h1bin/nph-tfsearch. The application of this algorithm to the sequence illustrated in FIG. 1 has revealed numerous motifs and their corresponding transcription factors. Accordingly, and in a more directed approach to the identification of GLP-2R promoter modulators, these binding interfaces can be targeted for either augmentation or interference. Compounds already known to modulate these specific interactions qualify as GLP-2R promoter modulators.

The screening and expression strategies described above, can similarly be exploited at the in vivo level, using transgenic animals carrying a transgene of interest under the expression control of the GLP-2R promoter. The effect in vivo of modulating agents on the function of the GLP-2R promoter can be determined when the agent is administered to a transgenic animal carrying a reporter gene under the expression control of the promoter. As is shown in the examples herein, for instance, the localization of cells mediating expression from the GLP-2R promoter are readily reported by the lacZ reporter gene product. Accordingly, agents that modulate promoter function can be identified following their administration to the transgenic animal, by variation in the levels of reporter gene product relative to control animals receiving no agent. Similarly, the effect of alterations in promoter structure can be revealed in order to map functional components and regions of the GLP-2R promoter.

In another aspect of the invention, the recombinant DNA expression constructs of the invention incorporate a gene of interest that encodes a therapeutic product, under the expression control of the GLP-2R promoter. Particularly suitable gene products are those therapeutic proteins that would usefully be produced in those tissues in which the endogenous GLP-2R promoter is active. As shown by the results herein, such tissues include the gastrointestinal epithelium, and particularly the jejunum as well as the ileum, colon, stomach and duodenum, as well as the lateral hypothalamus and particularly the amydylary-hippocampal nuclei, and the testes. The results herein also show that the endogenous GLP-2 promoter is active in regions of the Central Nervous System (CNS) including the cerebellum, medulla, pons, amygdala, cerebral cortex, pituitary and brainstem.

Gene products usefully expressed in the gastrointestinal epithelium are numerous, and include those proteins useful to elicit controlled growth thereof, including neurotensin, GLP-2 itself, growth hormone, insulin-like growth factors 1 and 2, glicentin, and keratinocyte growth factors 1 and 2. Still other useful gene products include those that are usefully secreted into the gastrointestinal tract, including anti-secretory factors, antibiotic peptides, tumoricidal proteins and the like. Proteins usefully expressed in the testes include the fertility hormones and releasing factors therefor. Proteins usefully expressed in the hypothalamus and hippocampal region of the brain are also numerous, and include the receptors and transporters for the various neurotransmitters such as serotonin, glycine, dopamine, NPY in its various forms, and glial and neurotrophic growth factors including nerve growth factor, neurotrophins 2 and 3, BDNF, and CNTF. In the case where secretion of the gene product is desired, the gene of interest encodes a secretable precursor of that protein. The naturally encoded secretion signals can be exploited for those products that are normally secreted, or a heterologous signal sequence, such as the GLP-2R secretion signal sequence, can be exploited in the routine manner.

Introduction of the recombinant DNA expression constructs can be achieved using techniques now established in the art. Transfection into cultured cells and tissues is readily achieved using standard in vitro methods that include bombardment, viral infection, electroporation, and microinjection, as well as calcium chloride mediated transformation, DEAE-dextran mediated transfection, and lipofection. Introduction of the recombinant DNA expression construct into living subjects, including mammals and particularly humans, can be achieved by dosing the subject with an appropriate formulation containing the receptor-encoding DNA coupled to the promoter appropriate for driving expression in the target tissue, as noted above. Subjects can, for instance, be treated with an oral dosage form of the receptor-encoding DNA construct, particularly for transfecting cells lining the gastrointestinal epithelium, using the procedures described generally by During et al. in Nature Medicine, 1998 Oct; 4(10): 1131, incorporated herein by reference. This treatment entails the formulation of the GLP-2 receptor-encoding construct as a liquid formulation comprising appropriate vehicle such as phosphate buffered saline, and, on a per mL basis, from about 10⁵ to 10⁸ e.g. about 10⁶, total infectious units of an adeno associated virus (AAV)-based construct carrying the construct. Delivery of the formulation can then occur perorally, using a volume appropriate for the size of subject undergoing transfection.

An alternative formulation useful for delivering the construct into a subject is described by Liptay et al. in Digestion 1998; 59(2): 142-7. In this formulation, which is particularly suitable for transfecting colonic cells in situ, cationic liposomes containing the construct in plasmid form are instilled, yielding colonic cells expressing the receptor transgene. Similarly, transfection of the colonic cells can be achieved in situ and following instillation of the construct by applying the DEAE dextran or calcium phosphate transformation methodologies.

As an alternative to peroral delivery of the rGLP-2 receptor transgene, the transgene can be injected directly into the tissue to be transfected, or the tissue to be modified can be transfected during culturing and then transplanted into the subject. It will thus be appreciated that the construct containing the gene of interest under the expression control of the GLP-2R promoter can be introduced using a variety of routes to achieve expression of the GLP-2 receptor in either cultured cells or tissues, or in subjects. Such treatments serve to elevate levels of the therapeutic expression product.

As a further alternative, the construct can be injected directly into a gametic or early embryonic form of the recipient animal, and then selected for maturation in the manner established for the creation of transgenic animals. Methods of making transgenic animals are well known to persons skilled in the art. Transgenic mice are produced essentially as described by Hogan et al., in MANIPULATING THE MOUSE EMBRYO 153-203 (Cold Spring Harbor Press 1986), or by Palmiter et al., Ann. Rev. Genet. 20:465-499 (1986). Additional guidance with respect to preparing transgenic animals appears in Houdebine, J. Biotechnology 34: 269-287 (1994), Lubon et al, Transfusion Medicine Reviews, Vol X (No. 2), 131-143 (1996), and TRANSGENIC ANIMALS: GENERATION AND USE (L. M. Houdebine, ed. 1997). Each of these publications is hereby incorporated by reference.

From the foregoing commentary and from the examples below, it will be apparent that the GLP-2R promoter is useful to drive expression of various gene products, including reporter genes useful to identify cells that sustain GLP-2R-mediated expression, to identify forms of the GLP-2R useful to drive that expression, and to identify agents that modulate expression from the GLP-2R promoter. The promoter of the present invention also is useful to drive expression of therapeutically efficacious gene products, in tissues within the GI tract, brain and testes, thereby to treat and protect those tissues from various diseases, disorders, and conditions.

### EXAMPLE 1

### Characterization of the promoter region of the murine GLP-2R gene.

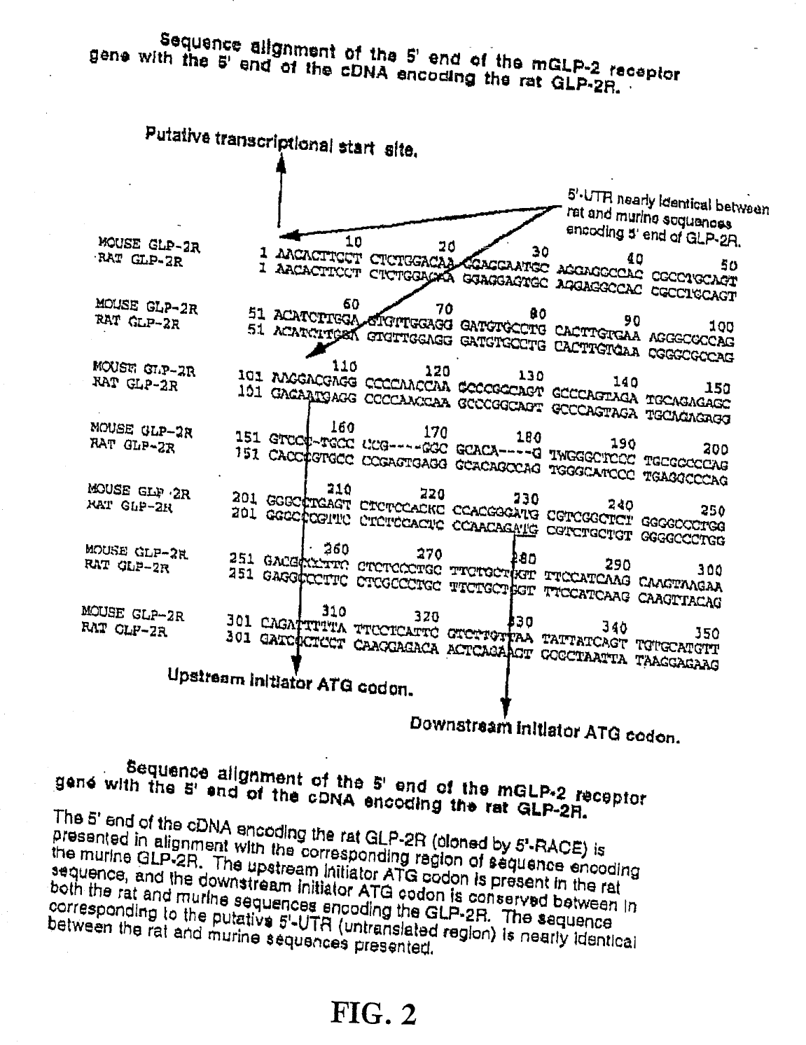

A) The identification of the transcriptional start site of the murine GLP-2R gene was localized by primer extension analysis and by carrying out 5′-RACE reactions in cDNA derived from rat brain. Although the cDNA encoding the rat GLP-2R gene has been cloned, the promoter region 5′ of the UTR has not been defined. The structure of the 5′ end of the first cDNA encoding the GLP-2R cloned from a rat hypothalamic library (Munroe et al., supra), contained approximately 25 bp of sequence corresponding to a putative 5′-UTR. Closely related receptors, including the mGlucagonR and the hGLP-1R however, contain larger 5′-UTRs. PCR products obtained from rat brain GLP-2R-specific primers in a 5′ rapid amplification of cDNA ends procedure were sequenced. The putative tss maps approximately to −220 bp (upstream of the downstream ATG) in the both the murine and rat GLP-2R genes. A comparison of the sequence encoding the putative 5′-UTR sequence in the murine gene to the 5′UTR sequence of the rat cDNA indicates that they are nearly identical (FIG. 2).

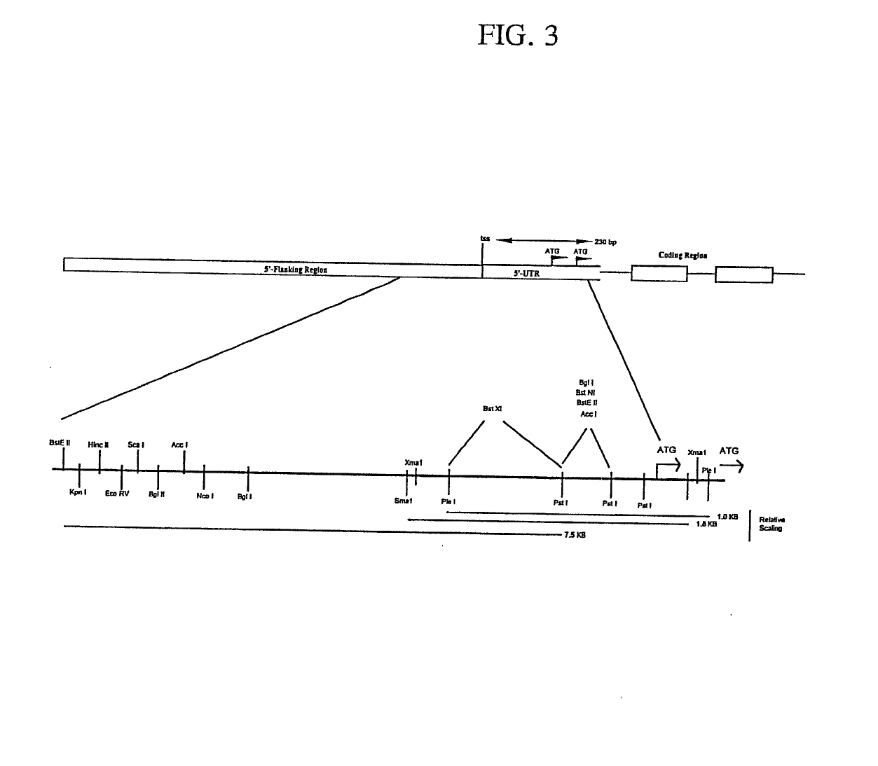

B) Amplification of the 5′ end of the cDNA encoding the rat GLP-2R gene (FIG. 3)

In order to isolate the entire 5′ end of the GLP-2R cDNA, 5′-RACE reactions were carried out using template cDNA derived from rat brain tissue (Clonetech). A predicted 500-bp fragment was cloned by nested PCR. In addition to the ‘downstream’ initiator ATG codon initially identified in the rat cDNA clone, a second initiator codon 126-bp upstream of the originally identified ATG is also present in the 5′-RACE product. Subsequent independent cloning confirmed that both the rat and human GLP-2R cDNA contains this 126-bp sequence extension that encodes for a unique 41-aa N-terminal moiety. Consensus sequence for vertebrate translation initiation sites, span the upstream initiator codon in both the rat and human cDNAs encoding the GLP-2R gene.

The relative positions of the oligonucleotide primers designed for the primer extension and 5′- RACE reactions are indicated in the shaded boxes of FIG. 2. The results of the primer extension reaction and position of the oligonucleotide primer used for the nested 5′-RACE reaction predicted a 500-basepair (bp) product. A 500-bp product was cloned and sequenced using 5′-RACE reactions. The 5′ end of the cDNA encoding the rat GLP-2R contains two putative translation initiation ATG codons, 126-bp apart. The putative tss (transcriptional start site) maps to approximately −230 bp upstream of the second initiator ATG codon in both rat and mouse transcripts encoding the GLP-2R.

C) Mapping the promoter region of the murine GLP-2 receptor gene.

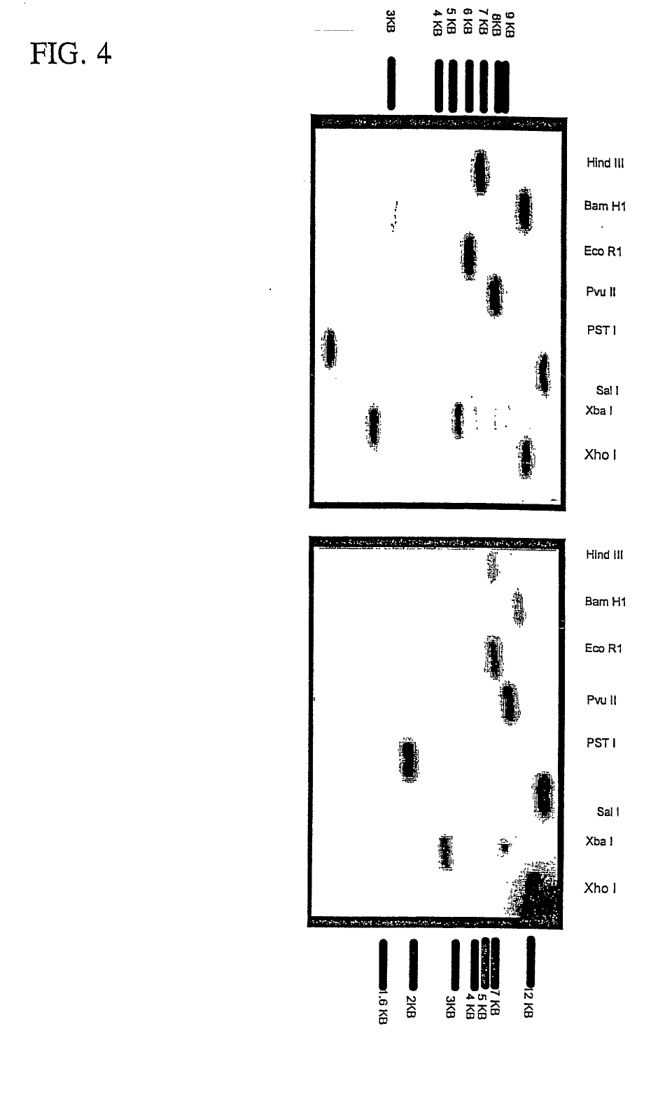

Mapping the promoter region of the murine GLP-2R gene was facilitated by Southern Blot analysis of a murine genomic clone known to encode the GLP-2R gene (FIG. 4). Southern Blot analysis was carried out with a labeled 213-bp Apa/Sma1 fragment from the 5′ end of the rat GLP-2R cDNA. The approximately 120-Kb murine genomic clone had been isolated with oligonucleotide primers complementary to the 5′ end of the rat GLP-2R cDNA and cloned into the BAC vector [Genome Systems Inc.] using techniques previously described. A 2-Kb Pst1 fragment of the murine GLP-2R gene was isolated from the BAC clone and was found to encode the putative translation initiation codon at its 5′end. Additionally, an upstream 1.6-Kb Sma1 fragment was identified in both the BAC clone and a 12-Kb BamH1 subclone (B7). A map of the promoter region of the murine GLP-2R gene was generated from differential hybridization of the BAC clone to 1) a 110-bp Pst1 /Sma1, 2) a 1.6-Kb Sma1 fragment of the murine GLP-2R gene, and 3) oligonucleotide primers downstream of the putative translation initiation sites.

### EXAMPLE 2

### Construction of a recombinant DNA expression construct

In this example, a GLP-2R promoter is linked operably for expression to a gene of interest, in this case the nls-lacZ reporter gene and then transfected to produce transgenic mice.

A 1.5-Kb Smal-Pstl genomic fragment corresponding to the 5′-flanking region of the GLP-2R gene was cloned from the BAC genomic clone known to encode the GLP-2R gene and was ligated into a promoterless-nls-lacZ encoding vector. The transgene DNA was gel purified using electroelution, and then microinjected using established techniques to produce proembryos that then were subjected to founder animal derivation. Founder animals were identified by PCR and Southern Blot analysis of genomic DNA isolated from tail biopsies. Initially eight putative founder animals were identified. Expression analysis in adult animals by RT-PCR detection of the transgene and histochemical detection of transgene β-galactosidase activity was initiated.

A recombinant DNA expression construct similar to that just described was also produced, but using an extended GLP-2R promoter region. Particularly, approximately a 9 kb Pstl-Smal genomic fragment corresponding to the 5′flanking region of the GLP-2R gene was cloned from the BAC genomic clone known to encode the GLP-2R gene and ligated with the 1.6 kb Smal-Pstl fragment at the compatible Smal ends. The 10.6 kb ligated product was then cloned into the promoterless-nls-lacZ encoding vector. The transgene DNA was gel purified using electroelution, and founder transgenic animals then were established.

### EXAMPLE 3

### Transgene Expression Analysis

Founder animals were identified by PCR and Southern Blot analysis of genomic DNA isolated from tail biopsies. For analysis, animals were sacrificed by CO₂ inhalation, and tissues of interest were removed immediately and snap frozen in liquid nitrogen. Total RNA was isolated from tissues using modified guanidine thiocyanate protocol. RNA concentrations were determined by spectrophotometry and approximately 2 μg of RNA was visualized using gel electrophoresis to assess RNA quality. Approximately 10 μg of RNA was pre-treated with DNAse I, divided into two (RT+, RT−) and reverse transcribed using Superscript II (Gibco). One μl of cDNA was PCR amplified using primers specific for the transgene (5′ oligonucleotide primer corresponds to the 5′-flanking region of the GLP-2R gene and 3′ oligonucleotide primer corresponds to the nls region of the transgene (gene of interest). PCR products were separated on an agarose gel, transferred to nitrocellulose by Southern Blot and hybridized using an P32-labeled oligo internal to the two oligo primers used in the PCR reaction.

Results were as follows.

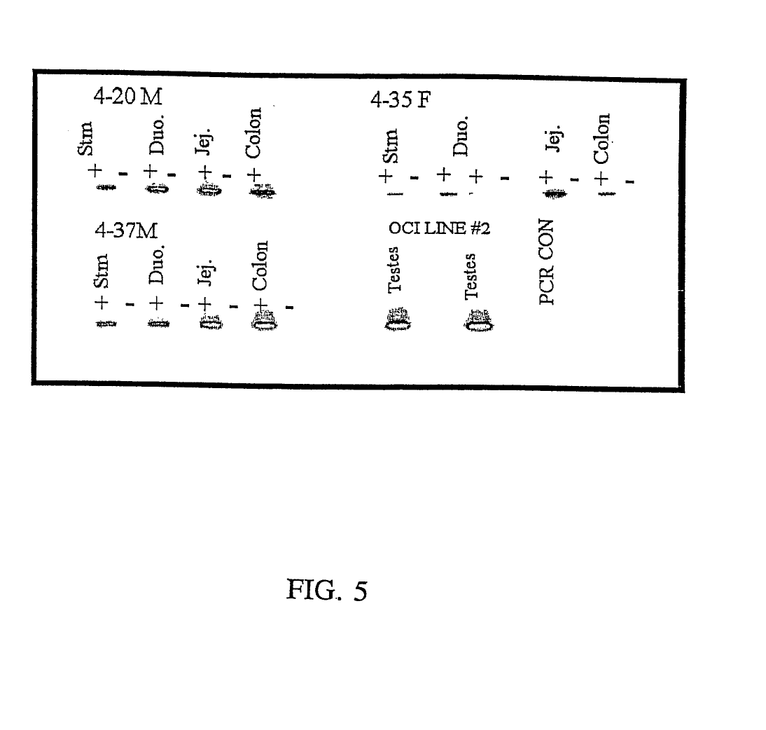

RT-PCR analysis reveal that reporter gene (nlsLacZ) expression directed under the control of the 1.5 kb GLP-2R promoter is present in the brain, stomach, duodenum, jejunum, ileum and colon of male and female adult (3-6 month) transgenic mice. Transgene expression has not been detected in any other tissue other than the testes. Results are shown in FIG. 5.

Animal tissues were also assessed histochemically. All gastrointestinal tissues taken for analysis were first syringe flushed with PBS. Sections of the GI tract approximately 1-2 cm in length were placed in 6-well tissue culture plates containing 4% paraformaldehyde/0.2% glutaraldeyhde in PBS fixative for 1 hr at room temperature. Brains (forebrain to the base of the brainstem) were removed from the animals intact and placed in 2% paraformaldehyde/0.2% glutaraldeyhde in PBS fixative for 1 hr at room temperature. All other tissues (heart, liver, testes, and lung) were cut in half longitudinally and otherwise treated similarly. After one hour, all tissues were rinsed three times with PBS, transferred to 15% sucrose/PBS for 4 hours to overnight at 4C., and then transferred to a 30% sucrose/PBS solution for 4 hours to overnight at 4C. for cryoprotection. All tissues were then frozen in dry ice vapor and stored at −80 degs Celsius. For sectioning, tissues were mounted on cryostat chuck, using O.C.T. compound (Tissue Tek) in a 25-30 degs Celsius chamber, were sectioned at 10 μm and thaw-mounted onto Superfrost Plus Slides (Fischer Scientific), and then were stored at −80 degs Celsius in slide boxes. Prior to X-Gal staining, slides were allowed to reach room temperature slowly and then were rinsed in PBS for 5 minutes. Slides were placed in a glass-staining dish which contained X-Gal solution (2.8 mM NaH₂PO₄, 7.2 mM Na₂HPO₄, 150 mM NaCl, 1 mM MgCl₂, 3.1 mM K4Fe(CN)₆, 3.1 mM K₃Fc(CN)₆, 3% Triton-X-100 and, at the last minute, 1 mg/ml X-Gal was added) at pH 7.2, wrapped in aluminum foil, and placed at 37° Celsius overnight. After X-Gal treatment, slides were rinsed in PBS twice for 5 minutes, counterstained with cosine and dehydrated in an ethanol series, and coverslipped with Permount (Sigma).

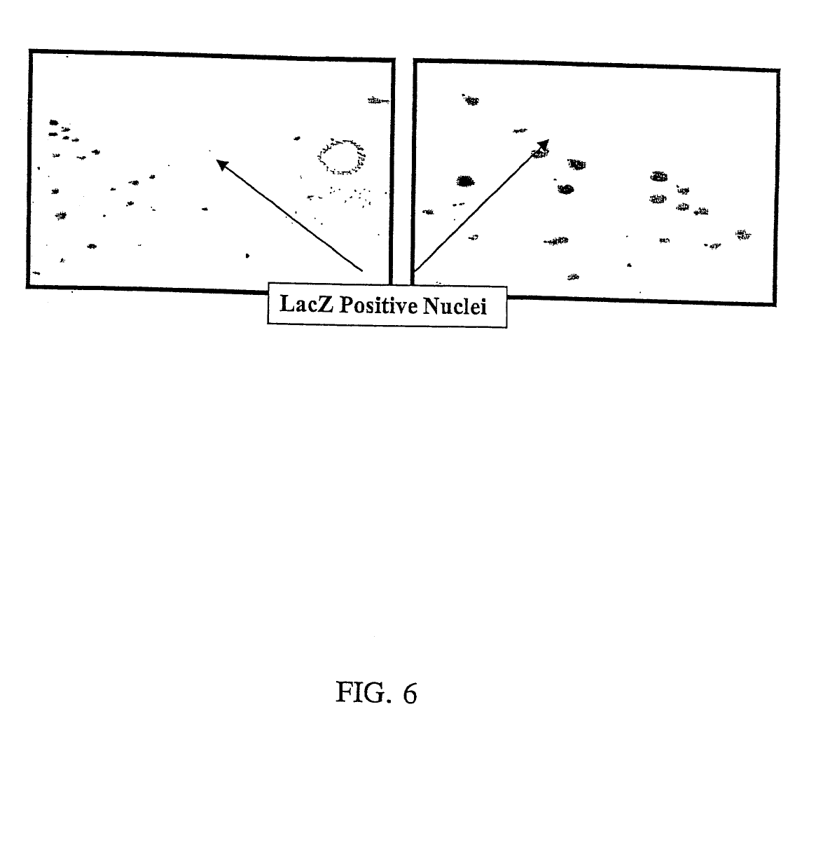

Brain-Histochemical detection of β-galactosidase activity, corresponding to transgene activity directed under 1.5 Kb of the GLP-2R promoter, is expressed in the lateral hypothalamus, and more specifically in regions of the cortical amgydala and hippocampal regions immediately adjacent to the surrounding amgydalary nucleus (FIG. 6). Staining has been specific to these regions in both male and female adult (3-6 months) animals.

The purpose of Examples 4-9 was to evaluate the biological function, mechanisms regulating control, and localization of GLP-2R expression using a combination of RT-PCR and immunohistochemistry analyses.

### EXAMPLE 4

The purpose of this example was to generate founder animals exhibiting germline transmission of the GLP-2R gene.

All animal experiments were approved and carried out strictly in accordance with the Canadian Council on Animal Care guidelines and the Animal Care Committee at the Toronto General Hospital, University Health Network, (Toronto, ON). Animals were allowed to acclimatize to the animal care facilities for at least one week prior to any experimental procedure.

Characterization of GLP-2R sequences and Transgene Construction

A genomic clone containing the 5′-flanking, 5′-untranslated and coding regions of the murine GLP-2R gene was isolated from a 129 SVJ mouse genomic library. To identify additional GLP-2R nucleotide sequences 5′-to the translation start site (Munroe et al., (1999) Proc. Natl. Acad. Sci. USA 96, 1569-1573), the 5′-end of the rat GLP-2R cDNA was generated and characterized using adaptor-modified complementary DNA from rat brain (Clontech Laboratories INC., Palo Alto, Calif.) in 5′-RACE (rapid amplification of cDNA ends) experiments.

Separately, a 1,516 bp (base-pair) fragment of the mouse GLP-2R gene was subcloned from the mouse genomic library (Incyte Genomics, St. Louis, Mo.), sequenced, and ligated immediately 5′- to a cDNA encoding LacZ with a nuclear localization signal (a gift from A. Nagy, Toronto, ON). The GLP-2R promoter-LacZ transgene was gel-purified and used for generation of transgenic mice.

In total, eight founder animals were identified by Southern blot and PCR analysis and mated with non-transgenic mice to determine germline transmission of the transgene. Three transgenic founder mice (designated #2, #3, #4) exhibited germline transmission, and were used to generate lines for further analysis of transgene expression.

### EXAMPLE 5

The purpose of this example was to conduct 5′-RACE experiments using a cDNA template from rat brain to identify 5′-untranslated sequences of the rat GLP-2R.

Nucleotide sequences at the 5′-end of the GLP-2R mRNA and gene:

FIG. 7a shows approximately 230 bp of sequence, including 104 bp of 5′-untranslated sequences corresponding to the 5′-end of the cDNA encoding the rat GLP-2R obtained from sequencing of RACE products. The vertical arrow indicates the 5′-end of the RACE product and the putative start of transcription. The 5′-UTR (untranslated region) is highlighted in bold letters. The coding sequence is presented in capital letters with the corresponding translated product presented above in capital bold letters.

FIG. 7b shows the organization of 5′-flanking and exon-1 sequences in the mouse GLP-2R gene compared to rat exon 1 and human GLP-2R 5′-flanking and 5′-untranslated sequences. Sequence identities are presented in bold print, and dashes indicate gaps introduced to maximize alignment. The DNA sequence is numbered from the putative transcription start site. Potential transcription factor binding regions in the putative promoter regions of the human and mouse genes are boxed. The predicted translation initiation codons in rat, mouse and human genes are underlined. The predicted translated product of the mouse gene is indicated above the nucleotide sequence. The vertical arrow indicates the predicted 5′-boundary of intron-1. The Pst I site, corresponding to the 3′-end of the GLP-2R promoter-LacZ transgene is indicated by arrowheads. The letters k, w are ambiguity codes where k=G or T, and w=A or T.

The GenBank accession numbers for the rat GLP-2R sequences derived from the RACE experiments is AF338223. The accession number for the nucleotide sequence of the GLP-2R mouse genomic clone is AF338224. The accession number for the previously published rat GLP-2R cDNA is AF105368. The accession number for GenBank sequences identified as corresponding to human GLP-2R genomic sequence is ACO69006. The accession number for the GenBank submission identified as corresponding to mouse GLP-2R genomic sequence is ACO16464.

FIG. 7c shows construction of the transgene achieved by inserting a 1.5-kb Sma I-Pst I fragment of the murine GLP-2R gene upstream of an nlsLacZ cDNA. The solid black box denotes the presence of GLP-2R 5′-untranslated sequences (5′-UTR) 5′-to the Pst I site shown in FIG. 7b.

Multiple RACE reaction products were consistently obtained that were ^{˜}500 bp in size. These products were cloned and sequence analysis demonstrated the presence of previously identified rat GLP-2R cDNA sequences (Munroe et al., (1999) Proc. Natl. Acad. Sci. USA 96, 1569-1573) and an additional 104 nucleotides of rat GLP-2R 5′-untranslated sequences upstream of the previously reported ATG (FIG. 7a).

Using a 213 bp Apa I/Sma I rat cDNA fragment containing 5′-coding sequences as a probe, a ^{˜}2 kb subclone was isolated from a BAC clone derived from a mouse genomic library. The DNA sequences of the mouse GLP-2R genomic subclone were aligned with the known rat GLP-2R cDNA sequence (Munroe et al., (1999) Proc. Natl. Acad. Sci. USA 96, 1569-1573) and with human GLP-2R genomic sequence we identified in GenBank, as shown in FIG. 7b.

The rat and mouse sequences exhibited 96% identity over the first 104 bp 5′- to the initiator ATG codon (rat); the mouse and human GLP-2R cDNAs exhibit 76% identity over this same region. Whereas the rat and human GLP-2R sequences contained an upstream ATG translation initiation site that would give rise to a GLP-2R protein containing an extra 41 amino acids at the N-terminus, a more distal ATG initiation codon, was identified in the mouse (FIG. 7b). Nevertheless, transfection studies using a rat GLP-2R cDNA that initiates translation from the downstream rat ATG corresponding to the position of the mouse ATG codon gives rise to a functional GLP-2 receptor. Munroe et al., (1999) Proc. Natl. Acad. Sci USA 96, 1569-1573; and Yusta et al., (1 999) J. Biol. Chem. 274(43), 30459-67. Hence the biological significance of the additional 41 amino acids predicted to be present in the rat and human but not the mouse GLP-2R sequence remains unclear.

Using the mouse genomic GLP-2R sequences, genomic sequences in GenBank were identified that shared 98% identity over 3,103 nucleotides with the murine GLP-2R genomic subclone isolated from BAC DNA. Analysis of the murine genomic GLP-2R sequence in GenBank also demonstrated the presence of a single translation initiation codon in the murine gene.

As the glucagon, GLP-1, and GLP-2 receptors are related members of a G protein coupled receptor superfamily (Sherwood et al., (2000) Endocr Rev 21(6), 619-70), the sequences of the 5′-untranslated and 5′-flanking regions of these 3 receptors were compared. Significant similarity was not found using base-pair matching over 5′-untranslated or putative promoter regions. No putative TATA or CAAT box sequences were identified in the mouse GLP-2R genomic sequences immediately 5′- to the end of the putative 5′-untranslated region. Computer analyses identified several potential transcription factor recognition sites (TFSEARCH ver.1.3; http://pdap1.trc.rwcp.or.jp/research/db/TFSEARCH.html) for CdxA, GATA-1, NF-Kappa B, and Sp1, as indicated in FIG. 7b.

Comparison of the 5′-flanking regions in the mouse and human GLP-2R genes reveal 70% identity over the first 200-nucleotides 5′ to the start of transcription, however sequence similarity diverged 5′- to this region.

Although the glucagon and GLP-1 receptor promoters have been characterized in cell transfection experiments in vitro (Lankat-Buttgereit et al., (1997) Peptides 18, 617-624; Galehshahi et al., (1 998) FEBS Lett 436(2), 163-8; Wildhage et al., (1999) Endocrinology 140(2), 624-31; and Portois, (1 999) J. Biol. Chem. 274(12), 8181-90), there is no information available regarding the transcriptional regulation of the genes encoding the glucagon, GLP-1, or GLP-2 receptors in vivo.

To identify GLP-2R regulatory sequences that direct GLP-2R gene transcription to various regions of the CNS, a ^{˜}1.5 kb fragment of the murine GLP-2R gene containing 5′-flanking and 5′-untranslated sequences upstream of the LacZ cDNA (FIG. 7c) were ligated, followed by generating several lines of GLP-2R promoter-LacZ transgenic mice. The expression of the endogenous murine GLP-2R gene with expression of the LacZ transgene in different regions of the murine CNS was then assessed and compared.

### EXAMPLE 6

The purpose of this example was to extract CNS tissues of male Sprague-Dawley rats and GLP-2R promoter-LacZ transgenic mice for later analysis.

CNS Tissue Dissections

Male Sprague-Dawley rats (300-500 g) or GLP-2R promoter-LacZ transgenic mice were sacrificed by CO₂ inhalation and quickly decapitated. The brains were rapidly removed and placed ventral side up on a chilled glass plate. The pituitary glands were also removed and frozen in liquid nitrogen. The amygdala, cerebral cortex, cerebellum, pons/midbrain, and medulla were dissected and frozen in liquid nitrogen.

The amygdala was dissected by first producing a 3 mm thick coronal section by making a coronal cut at the optic chiasm and at the posterior edge of the mammillary bodies. A cut connecting the rhinal fissures formed the dorsal boundary of the amygdaloid block and cuts made continuous with the lateral ventricles to the lateral hypothalamic sulci formed the medial boundaries of the amygdaloid blocks respectively. The cerebral cortex was also taken from this coronal section and consisted primarily of parietal and frontal cortex. The cerebellum was removed and a coronal cut was made at the posterior edge of the pons. The neural tissue posterior to this cut comprised the medulla, which also contained the anterior most portion of the spinal cord. The midbrain block, which also included the pons, extended from the posterior edge of the mammillary bodies to the posterior edge of the pons, with the cerebellar and cerebral cortices, hippocampus, and amygdala removed.

### EXAMPLE 7

The purpose of this example was to isolate RNA and conduct reverse transcriptase PCR analysis of the CNS tissues obtained in Example 6.

RNA isolation and reverse transcription polymerase chain reaction (RT-PCR) analyses

Total RNA was isolated from CNS tissues using Trizol™ reagent (Gibco BRL, Toronto, Ontario) and from peripheral tissues using a modified guanidinium thiocynate procedure and dissolved in ribonuclease-free water. RNA integrity was assessed on a 1%-(wt/vl)- agarose gel containing formaldehyde and visualized on a UV-transilluminator (Fischer Scientific, Montreal, Quebec) using ethidium bromide staining. For RT-PCR experiments RNA samples were treated with DNase I (GibcoBRL), primed with random hexamers (Gibco BRL), and reverse-transcribed with SUPERSCRIPT™ II Reverse Transcriptase (Gibco BRL). To control for contamination, reactions were also carried out in the absence of SUPERSCRIPT™. Following first-strand cDNA synthesis, samples were treated with Ribonuclease H (MBI Fermentas, Vilnius, Lithuania) to remove RNA.

For subsequent PCR amplification, first strand cDNA was used as template. Oligonucleotide primer pairs, annealing temperature, and cycle number for PCR amplification were as follows:

(1) for rat GLP-2R: 5′-TTGTGAACGGGCGCCAGGAGA-3′ and 5′-GATCTCACTCTCTTCCAGAATCTC-3′ were annealed at 65° C. for 40 cycles;

(2) for mouse GLP-2R: 5′-CTGCTGGTTTCCATCAAGCAA-3′ and 5′-TAGATCTCACTCTCTTCCAGA-3′ were annealed at 65° C. for 30 cycles;

(3) for rat GAPDH: (glyceraldehyde-3-phosphate dehydrogenase) 5′-TCCACCACCCTGTTGCTGTAG-3′ and 5′-GACCACAGTCCATGACATCACT-3′ were annealed at 60° C. for 30 cycles; and

(4) for GLP-2R-LacZ transgene: 5′-CGCTGATTTGTGTAGTCGGTT-3′ and 5′-CTTATTCGCCTTGCAGCACAT-3′ were annealed at 63° C. for 40 cycles.

The expected PCR product for the mouse and rat GLP-2R cDNA is ^{˜}1.6 kb, corresponding to full length GLP-2R. Munroe et al., (1 999) Proc. Natl. Acad. Sci. USA 96, 1569-1573; and Yusta et al., (2000) Gastroenterology 119(3), 744-755. The predicted LacZ PCR product is ¹⁸ 580 bp, and for rat GAPDH the expected PCR product is ^{˜}450 bp. To control for non-specific amplification, PCR reactions were also carried out in the absence of first strand cDNA.

Following amplification, PCR products were separated by gel electrophoresis, transferred onto a nylon membrane (GeneScreen, Life Technologies), and hybridized with a ³²P-labeled: (1) internal cDNA probe for rat GLP-2R (Munroe et al., (1999) Proc. Natl. Acad. Sci. USA 96, 1569-1573; and Yusta et al., (2000) Gastroenterology 119(3), 744-755), or (2) an internal LacZ oligonucleotide (5′-TCAGGAAGATCGCACTCCAGC-3′), or (3) an internal cDNA probe for rat GAPDH (Piechaczyk et al., (1984) Nucleic Acids Res. 12(18), 6951-63). Following hybridization, membranes were washed stringently and hybridization signals were quantified on a STORM 840-phosphorimager (Molecular Dynamics, Sunnyvale, Calif.) using ImageQuaNT™ software, Version 5.0, Molecular Dynamics.

Results

RT-PCR analysis of endogenous GLP-2 receptor and GLP-2R promoter-LacZ transgene expression in adult mouse tissues.

FIG. 8a shows the RT-PCR analysis of (i) transgene (nlsLacZ) expression in two different lines of 1.5-kb GLP-2R promoter-LacZ transgenic mice (lines #4, and #3) and (ii) endogenous GLP-2R mRNA transcripts in gastrointestinal tissues.

FIG. 8b shows the RT-PCR analysis of endogenous GLP-2R, GLP-2R promoter-LacZ (nlsLacZ), and GAPDH mRNA transcripts in structures isolated from the CNS of GLP-2R promoter-LacZ transgenic mice.

FIG. 8c shows the analysis of the tissue-specificity of transgene expression in liver, kidney, lung, spleen, and heart from 1.5-kb GLP-2R promoter-LacZ transgenic mice. PCR control designates reaction reactions carried out in the absence of cDNA, whereas RT- control indicates reactions carried out in the absence of reverse transcriptase. + and − denotes the presence or absence of first strand cDNA.

FIG. 8d shows the analysis of transgene expression in frozen histological sections (kidney and brain) from 1.5-kb GLP-2R promoter-LacZ transgenic mice and littermate control mice incubated with β-galactosidase antiserum.

Consistent with the highly tissue-specific expression of the endogenous rat and human GLP-2R in the GI tract (Munroe et al., (1999) Proc. Natl. Acad. Sci. USA 96, 1569-1573; and Yusta et al., (2000) Gastroenterology 119(3), 744-755), both LacZ transgene and endogenous GLP-2R mRNA transcripts were detected in stomach (data not shown), and in both the small and large bowel (FIG. 8a). Similarly, GLP-2R transcripts were observed in several regions of the murine CNS (FIG. 8b). Endogenous GLP-2R mRNA and transgene-derived LacZ transcripts were identified in the cerebellum, medulla, pons, amygdala, and cerebral cortex of GLP-2R promoter-LacZ mice (FIG. 8b). In contrast, GLP-2R mRNA transcripts, but not LacZ mRNA transcripts, were detected in the pituitary gland.

The results of previous studies localized rat and human GLP-2R transcripts and immunoreactive protein principally to the gastrointestinal tract and CNS (Munroe et al., (1999) Proc. Natl. Acad. Sci. USA 96, 1569-1573; and Yusta et al., (2000) Gastroenterology 119(3), 744-755). Consistent with the tissue specificity of endogenous GLP-2R expression, the GLP-2R promoter-LacZ transgene was not expressed in the liver, kidney, spleen, or heart of transgenic mice (FIG. 8c).

The specificity of transgene expression was further illustrated by demonstrating that tissues which did not contain transgene-derived mRNA transcripts also did not contain LacZ positive cell types (FIG. 8d). Furthermore, additional evidence for the regional specificity of transgene expression in the CNS derives from analysis of numerous coronal sections of frozen brain tissue from GLP-2R promoter-LacZ transgenic mice, the majority of which did not exhibit any β-galactosidase-like immunopositivity when incubated with a β-galactosidase-specific antisera (FIG. 8d and data not shown). Similarly, the majority of coronal sections of frozen brain tissue incubated with GLP-2R antiserum were also immunonegative.

### EXAMPLE 8

The purpose of this example was to conduct immunocytochemistry analyses of CNS tissues from Male Sprague-Dawley rats (300-500 g) or GLP-2R promoter-LacZ transgenic mice.

Immunocytochemistry

Sprague-Dawley rats or mice were deeply anesthetized following intraperitoneal injections of sodium pentobarbital. Following transcardial perfusion with 0.9% sodium chloride, animals were perfused with 4% neutral-buffered formalin for ^{˜}15 minutes. Brains were removed and post-fixed at room temperature for 4 hrs or overnight at 4° C. Brains were then cryopreserved overnight in a 20% sucrose in PBS (phosphate-buffered saline) gradient at 4° C., frozen slowly in dry ice vapor, and either stored at −80° C. or sectioned immediately at 10 um on a cryostat. All sections were collected and thaw-mounted onto Superfrost plus slides (Fischer Scientific, Montreal, Quebec).

For detection of β-galactosidase immunopositive cells, slides were incubated for 4 hrs at 37° C. in 1:8000 dilution of polyclonal anti-β-Galactosidase antiserum (ICN Pharmaceuticals Inc., Costa Mesa, Calif.). Polyclonal GLP-2R antiserum (1:800) that recognizes the GLP-2R but not the glucagon, Glucose-dependent inhibitory polypeptide, or GLP-1 receptors was a gift from NPS Allelix Corp. (Mississauga, Canada). To control for non-specific immunopositivity, GLP-2R antiserum was also preabsorbed overnight at 4° C. in the presence of recombinant GLP-2R immunogen and ICC (immunocytochemistry) studies were carried out with: (a) GLP-2R antiserum (b) preimmune serum (c) antibody diluent alone, or (d) preabsorbed GLP-2R antiserum. All slides were counterstained in haematoxylin.

Microscopy

All slides were visualized and captured using a video camera (JVC) with a ½″ chip device adapted (0.63X c-mount) to a light microscope Leica (Leica Ltd, Cambridge). Magnification is reported as the objective magnification multiplied by the c-mount magnification multiplied by the electronic magnification (electronic magnification was corrected for by dividing the diagonal of the image captured by the camera chip size).

Results

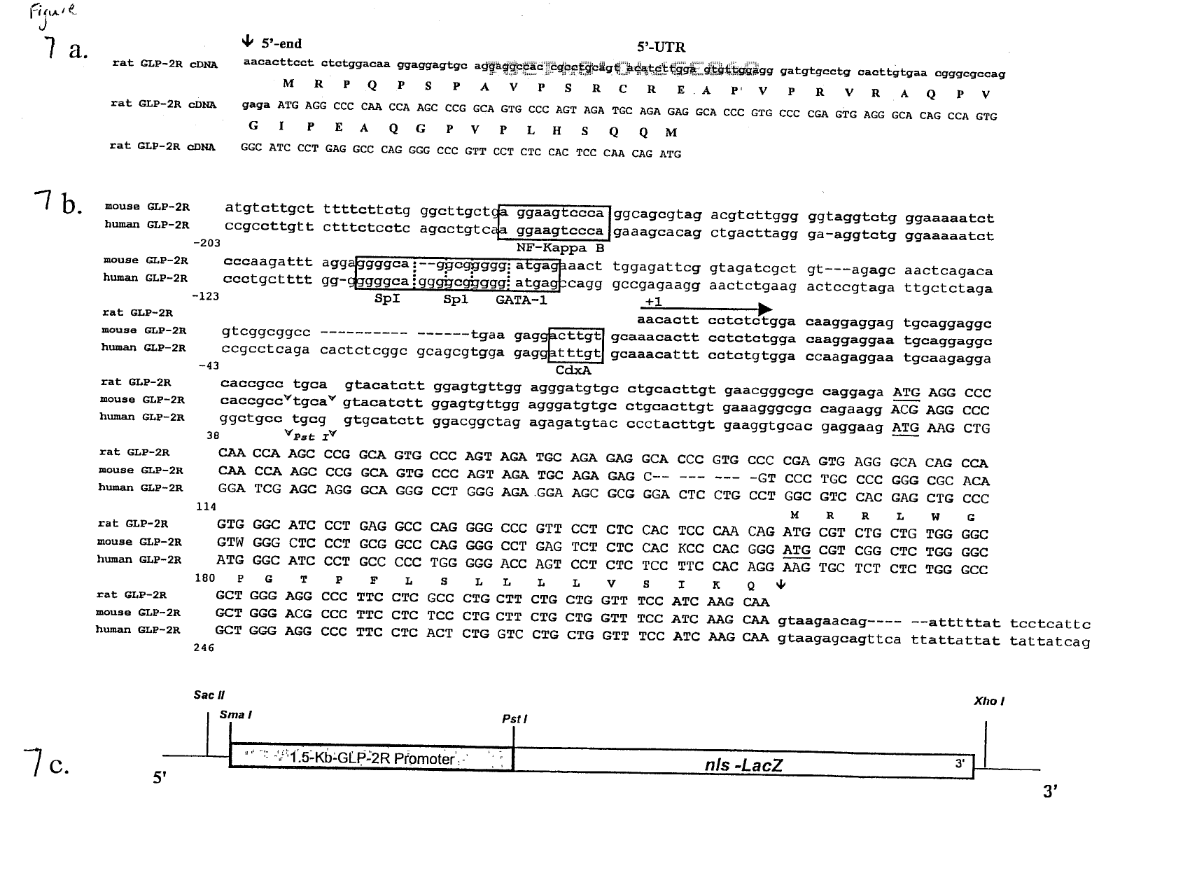

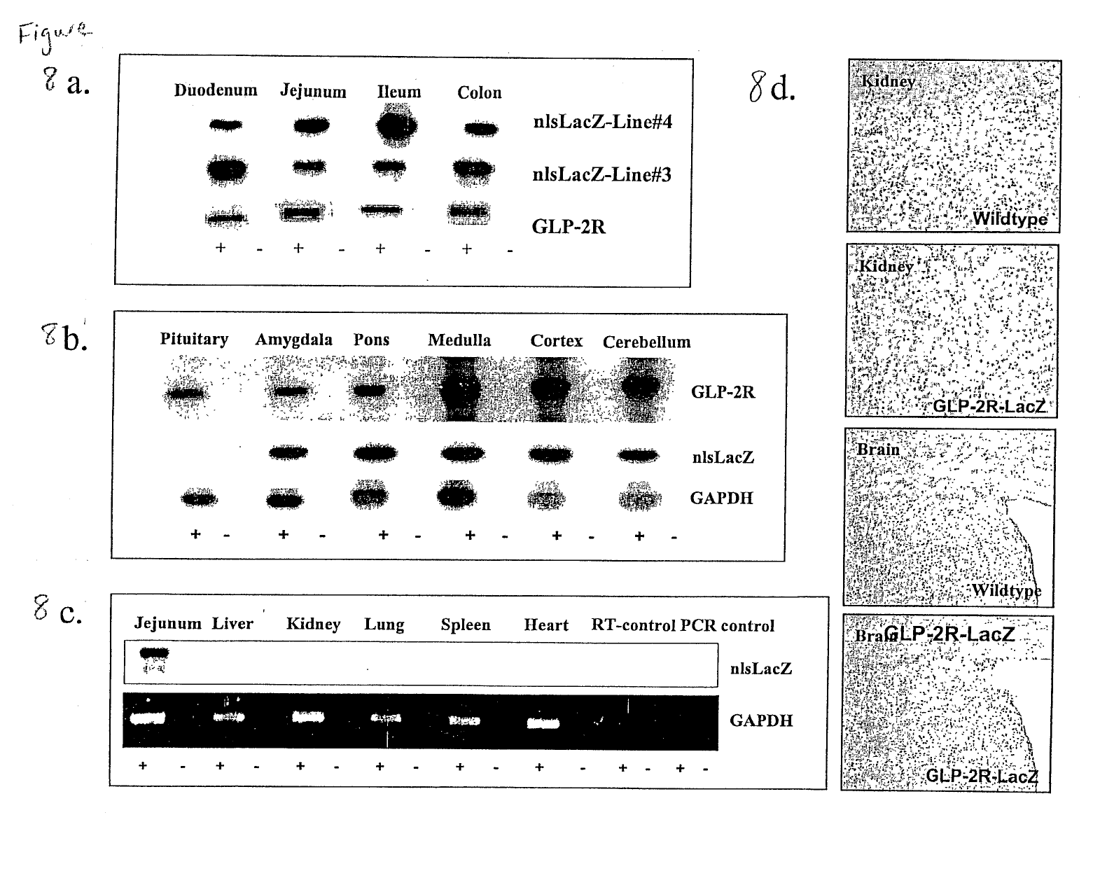

Analysis of endogenous GLP-2R and GLP-2R promoter-LacZ transgene expression in the cerebellum: Frozen coronal sections of formalin-perfused rat (FIGS. 9a-b), and mouse (FIGS. 9c-d) cerebellum were exposed to GLP-2R antiserum or to preimmune serum to detect the specific presence of GLP-2R positive cells. Frozen coronal sections of formalin-perfused GLP-2R promoter-LacZ (FIG. 9e) and littermate control (FIG. 9f) mouse cerebellum were incubated with a polyclonal antibody generated against the β-galactosidase enzyme to detect specific nuclear LacZ expression in the transgenic cerebellum. Histology presented corresponds approximately to the coordinates in FIG. 91 in the atlas of Franklin and Paxinos (Franklin et al., (1997) The mouse brain in sterotaxic coordinates, Academic Press, San Diego).

To localize specific regions and cell types within the rat and mouse CNS that express the endogenous GLP-2R, immunopurified antiserum directed against the carboxy-terminal region of the rat GLP-2R was used. The specificity of this antiserum has been previously characterized (Yusta et al., (2000) Gastroenterology 119(3), 744-755). The antiserum specifically recognizes the GLP-2R as a single major product of ^{˜}72 kDa on Western blot analysis and does not exhibit cross-reactivity against the related glucagon or GLP-1 receptors as demonstrated by the lack of immunopositivity in histological sections of rat liver or pancreas incubated with the GLP-2R antiserum (Id.).

GLP-2R immunoreactive cells were observed in the Purkinje layer of the rat cerebellum with no staining detected in other cell types throughout the cerebellum (FIG. 9a). GLP-2R immunoreactive neurons were also detected in the Purkinje cell layer of the murine cerebellum (FIG. 9c). In contrast, adjacent sections incubated with preimmune serum (FIG. 9b) or without primary antibody did not exhibit immunopositivity in the Purkinje cell layer. Similarly, preabsorption of GLP-2R antiserum with recombinant GLP-2R protein completely eliminated GLP-2R immunoreactivity in the cerebellum.

Analysis of transgene expression in GLP-2R promoter LacZ transgenic mice demonstrated nuclear LacZ immunopositivity in the Purkinje cell layer of the cerebellum, consistent with the presence of a nuclear localization signal in the modified LacZ coding sequence (FIG. 9e). In contrast, LacZ+ cells were not detected in the cerebellum of age-matched non-transgenic littermate controls (FIG. 9f). Purkinje neurons exhibiting positivity for the native GLP-2R in rat and mouse cerebellum detected with antiserum against the endogenous GLP-2R were comparatively more abundant than the number of LacZ immunopositive Purkinje neurons identified in transgenic mouse cerebellum.

### EXAMPLE 9

The purpose of this example was to conduct histochemical analyses of CNS tissues from Male Sprague-Dawley rats (300-500 g) or GLP-2R promoter-LacZ transgenic mice.

Histochemical Analysis

Brains were isolated from mice and placed in 2% paraformaldehyde/0.2% glutaraldehyde in PBS fixative for 1 hr at room temperature. Sixty minutes later, brains were rinsed in PBS and transferred to 4° C. in 15% sucrose in PBS solution for 4 hours to overnight, and subsequently to a 30% sucrose in PBS solution for 4 hours to overnight for cryopreservation. Brains were then frozen in dry ice vapor and stored at −80° C.

Tissues were sectioned at 10 um in a −25-30° C. cryostat, and subsequently thaw-mounted and stored at −80° C. Prior to 5-bromo-4-chloro-3-indolyl-β-D-galactopyranoside (X-GAL, Bioshop Canada, Burlington Ontario) staining, slides were slowly warmed to room temperature and rinsed in PBS. Slides were treated with X-GAL solution overnight at 37° C. Following treatment with X-GAL solution, slides were rinsed in PBS, counterstained with eosin and dehydrated in an ethanol series.

Microscopy

All slides were visualized and captured using a video camera (JVC) with a ½″ chip device adapted (0.63X c-mount) to a light microscope Leica (Leica Ltd, Cambridge). Magnification is reported as the objective magnification multiplied by the c-mount magnification multiplied by the electronic magnification (electronic magnification was corrected for by dividing the diagonal of the image captured by the camera chip size).

Results

Histological analysis of GLP-2R and GLP-2R promoter-LacZ transgene expression in the hippocampus and dentate gyrus.

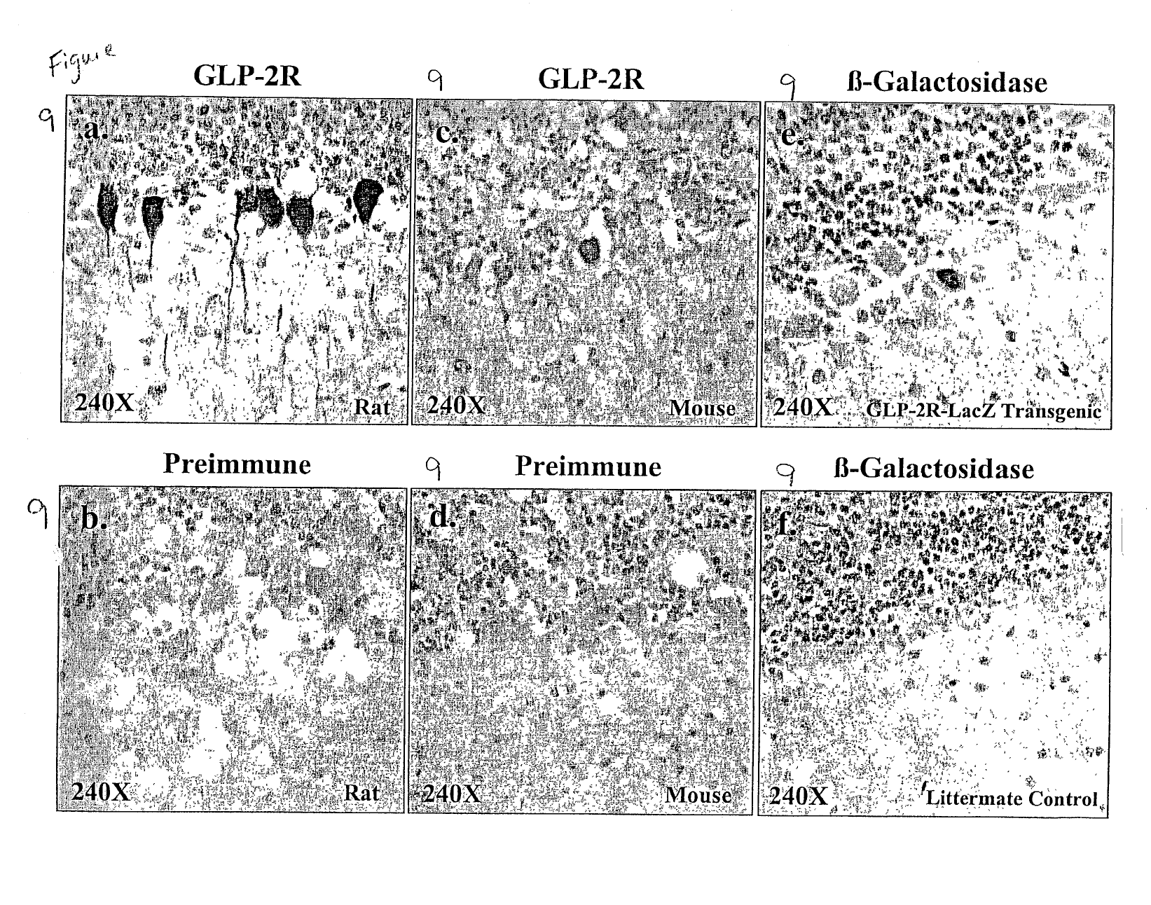

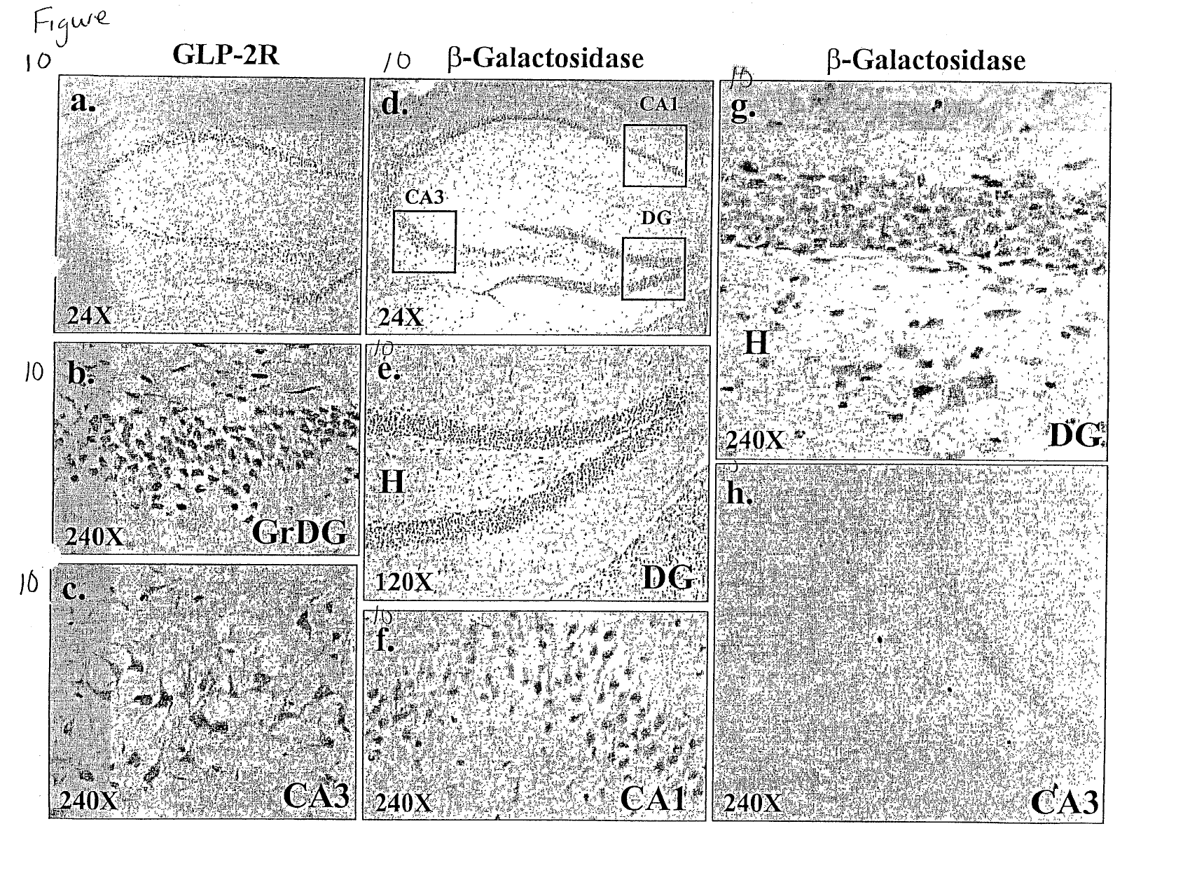

FIGS. 10a-c: Frozen coronal sections of perfused rat brain were incubated with GLP-2R antiserum to detect the presence of GLP-2R positive cell types in the hippocampus and dentate gyrus. Endogenous GLP-2R immunostaining in (FIG. 10a) rostral hippocampus, (FIG. 10b) granular layer of dentate gyrus, and (FIG. 10c) CA3 field of hippocampus.

FIGS. 10d-h: Frozen coronal sections of perfused GLP-2R promoter-LacZ mouse brain were incubated with β-galactosidase antibody to identify transgene positive nuclei in the transgenic hippocampus and dentate gyrus. FIG. 10d: The boxed regions correspond to areas containing LacZ+ positive cells including the transgenic dentate gyrus (FIGS. 10e and g) and the CA1 (FIG. 10f) field of the transgenic hippocampus using β-galactosidase immunocytochemistry. FIG. 10h: Caudal CA3 field of the transgenic hippocampus using X-GAL histochemical analysis. The relative magnifications, 24-240×, are shown in each panel. Histological regions represented in (FIGS. 10a-c) correspond approximately to the coordinates in FIG. 40-41; in (FIGS. 10d, e, and g) histological regions correspond approximately to FIG. 43; in (FIG. 10f) histological regions correspond approximately to FIG. 53; and in (FIG. 10h) histological regions correspond approximately to FIG. (59) in the atlas of Franklin and Paxinos. (GrDG=granular layer of dentate gyrus: DG=dentate gyrus; H=hilus region; cp=basal cerebral peduncle.)

As both GLP-2R and GLP-2R promoter-LacZ RNA transcripts were detected in extrahypothalamic regions of the rat CNS (FIG. 8b), these regions were next examined for GLP-2R immunoreactivity. GLP-2R immunoreactive cells were detected in the hippocampus and dentate gyrus of the rat CNS (FIGS. 10a-c). Numerous GLP-2R immunoreactive cells were observed in the pyramidal cell layer of the rostral rat hippocampus, including the CA1, CA2, CA3 fields; in the caudal hippocampus GLP-2R+ cells were also present in the CA3 fields. GLP-2R immunoreactive staining was also observed in scattered cell types located in the granular layer and polymorphic hilus region of the rat dentate gyrus. In contrast, preabsorption of antiserum with recombinant GLP-2R or the use of preimmune antiserum did not result in detectable GLP-2R immunopositivity in adjacent serial sections (data not shown).

Following detection of GLP-2R immunopositivity in the rat hippocampus and dentate gyrus, these same structures were next examined in the CNS of GLP-2R promoter-LacZ transgenic mice. Nuclear LacZ immunopositivity was clearly visible within similar cell types (that also exhibited endogenous GLP-2R immunoreactivity) positioned in the hippocampus and dentate gyrus in GLP-2R promoter-LacZ transgenic mice (FIGS. 10d-h). Corresponding age-matched littermate non-transgenic control sections did not exhibit LacZ immunopositivity under identical staining conditions.

In contrast to the extent of endogenous GLP-2R immunoreactivity observed in the pyramidal layers of the rat hippocampus, LacZ immunopositive cells in the CA1, CA2, and CA3 fields of the transgenic hippocampus were less abundant, with the highest density of positive cells observed in the CA3 field (FIG. 10h). Nevertheless, β-galactosidase positive cells were dense in the granular layer of the transgenic dentate gyrus (FIG. 10g). Cells with nuclear β-galactosidase staining were also observed in the polymorphic hilus region of the transgenic dentate gyrus, consistent with the distribution of GLP-2R positive cells observed in the rat dentate gyrus using GLP-2R antiserum.

Histological analysis of GLP-2R promoter-LacZ expression in the amygdaloid, hypothalamic and thalamic nuclei of transgenic mice.

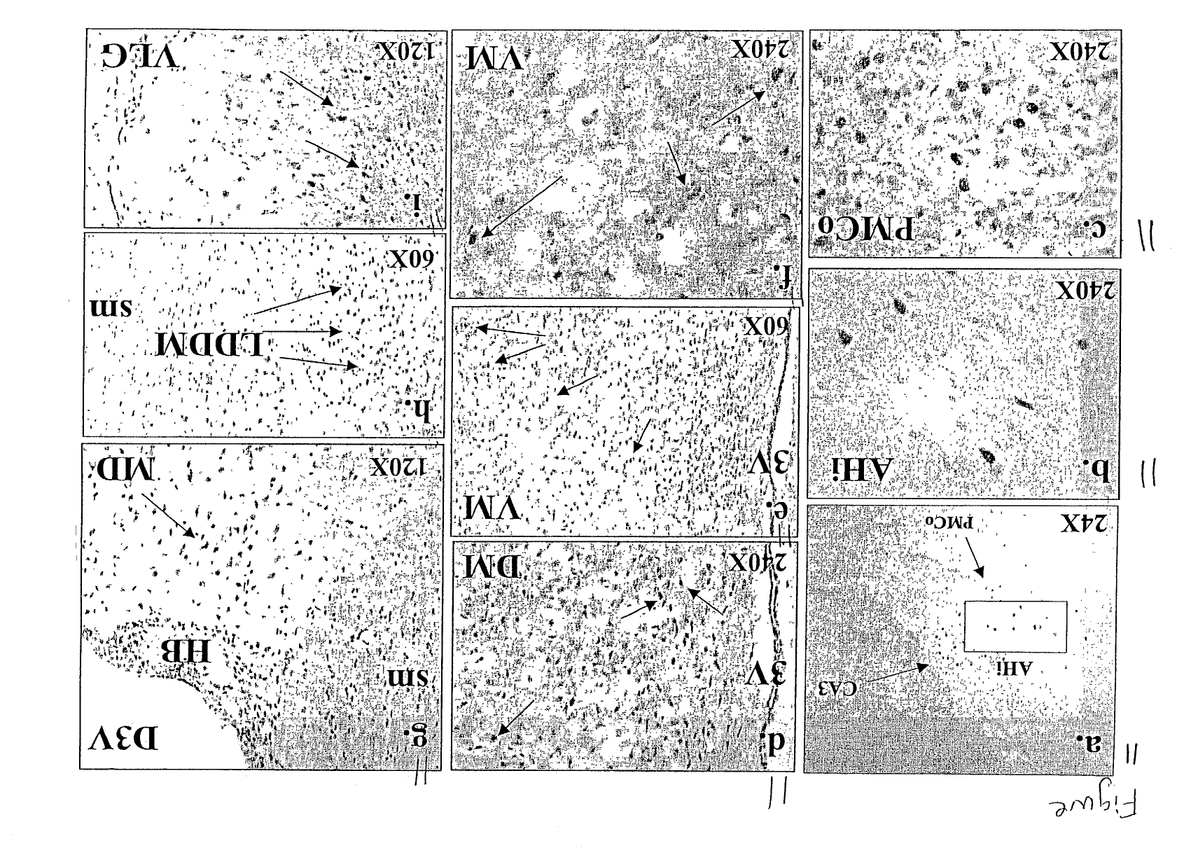

FIG. 11a: Histochemical analysis of LacZ positivity in the amygdaloid nucleus of GLP-2R promoter-LacZ transgenic mice using X-GAL staining. The boxed area corresponds to LacZ+ nuclei in the amygdalohippocampal area and the arrows point to LacZ+ nuclei in the caudal CA3 region of the hippocampus and the posteromedial cortical amygdaloid nucleus.

FIGS. 11b and c: Immunocytochemical analysis of the amygdalohippocampal area (FIG. 11b) and the posteromedial (FIG. 11c) cortical amygdaloid nucleus using an antibody directed against the β-galactosidase enzyme.

FIGS. 11d-f: Immunocytochemical analysis of the hypothalamus of GLP-2R promoter-LacZ transgenic mice using β-galactosidase antiserum, specifically in the dorsomedial nucleus (FIG. 11d) and in the ventromedial nucleus (FIG. 11e) 60× and (FIG. 11f) 240× magnification.

FIGS. 11g-i: Immunocytochemical analysis of thalamic nuclei in GLP-2R promoter-LacZ transgenic mice using β-galactosidase antiserum, specifically in the mediodorsal nuclei (FIG. 11g) the dorsomedial part of the laterodorsal thalamic nucleus (FIG. 11h) and the ventrolateral geniculate nucleus (FIG. 11i). The arrows point to LacZ+ nuclei. The relative magnifications 24-240×, are indicated.

Histology presented in (FIGS. 11a-c) corresponds approximately to the coordinates in FIGS. 51-52; in (FIG. 11d-f) corresponds approximately to FIGS. 46-47; in (Figure g) corresponds approximately to FIG. 38; in (Figure h) corresponds approximately to FIG. 41; and in (FIG. 11i) corresponds approximately to FIGS. 46-47 in the atlas of Franklin and Paxinos. (AHi=amygdalohippocampal area; PMCo=posteromedial cortical amygdaloid nucleus; cp=basal cerebral peduncle; DM=dorsomedial hypothalamic nucleus; VM=ventromedial hypothalamic nucleus; 3V=3^{rd} ventricle; D3V=dorsal 3^{rd} ventricle; HB=habenular nucleus; sm=stria medullaris; MD=mediodorsal thalamic nucleus; LLDM=laterodorsal thalamic nucleus; VLG=ventrolateral geniculate nucleus.)

Examination of the CNS in GLP-2R promoter-LacZ transgenic mice revealed additional structures that exhibited intense β-galactosidase activity. The amygdala contained a number of LacZ positive cells detected bihemispherically by both histochemical and immunocytochemical staining (FIGS. 11a-c). Positive cell staining was principally restricted to the posterolateral and posteromedial cortical amygdaloid nucleus and to the amygdalohippocampal area. The lateral and medial amygdaloid nuclei did not contain positive staining.

The results of previous GLP-2R in situ hybridization studies demonstrated restricted hypothalamic GLP-2R expression exclusively in the caudal part of the rat dorsomedial nucleus of the hypothalamus. Tang-Christensen et al., (2000) Nat. Med 6(7), 802-7. Immunocytochemical analysis of the transgenic hypothalamus revealed occasional rare LacZ positive nuclei of the dorsomedial nucleus (FIG. 11d). A few rare nuclear LacZ+ nuclei were also detected throughout the ventromedial hypothalamic nucleus in transgenic mice (FIGS. 11e and f).

Neural populations exhibiting nuclear β-galactosidase activity were also localized to the thalamic nuclei of GLP-2R promoter-LacZ transgenic mice (FIGS. 11g and h). Nuclear LacZ immunopositivity was observed in select cells in the mediodorsal thalamic nucleus, and in the ventrolateral and dorsomedial part of the laterodorsal thalamic nucleus. Immunopositivity was also observed in the ventrolateral geniculate nucleus with the majority of positive cells in the parvocellular regions (FIG. 11i). A few immunopositive cells were identified in the caudate putamen, dorsal fornix and septofimbrial nucleus of GLP-2R promoter-LacZ mice. Nuclear β-galactosidase positivity was also detected in cells at the base of the corpus callosum in the ventral endopiriform nucleus and in the piriform cortex (data not shown).

Although GLP-2 receptor expression was previously localized exclusively to the dorsomedial hypothalamic nucleus in rats by in situ hybridization (Tang-Christensen et al., (2000) Nat. Med 6(7), 802-7), the data of examples 4-9 extends these findings by demonstrating expression of the GLP-2R in multiple CNS regions of rats and mice by both RT-PCR and immunohistochemical analyses.

Furthermore, the demonstrated specificity of the GLP-2R antiserum (Yusta et al., (2000) Gastroenterology 119(3), 744-755), taken together with the colocalization of endogenous GLP-2R immunoreactivity and nuclear localization of LacZ transgene expression in multiple CNS regions, provides additional evidence supporting a more widespread GLP-2R expression pattern extending beyond the hypothalamus. The findings of extrahypothalamic GLP-2R expression are consistent with a recent report localizing rat GLP-2R mRNA transcripts to the hypothalamus, cortex, midbrain, hippocampus, and striatum. White et al., (2000) 82nd Annual Meeting of the Endocrine Society Toronto, Ontario, 271A1115.

The mechanisms regulating expression of the receptors for glucagon, GLP-1, and GLP-2 have not been extensively examined. Although promoter sequences directing expression of the glucagon and GLP-1 receptor sequences have been analyzed in cell-based transfection studies (Lankat-Buttgereit et al., (1997) Peptides 18, 61 7-624; and Portois et al., (1999) J. Biol. Chem. 274(12), 8181-90), the DNA regulatory sequences mediating tissue-specific control of these receptor genes in vivo have not yet been identified. Furthermore, the analysis of the 5′-ends of the receptor coding regions, 5′-untranslated, and 5′-flanking region DNA sequences (described above) does not reveal significant shared nucleotide identity across the glucagon, GLP-1, or GLP-2 receptors, providing indirect evidence for the evolution of distinct control mechanisms regulating the transcription of each receptor gene.

The experimental results extend the previously reported GLP-2 receptor sequence at the 5′-end and identify both the 5′-untranslated region and the location of intron 1. Furthermore, functional evidence is provided for the transcriptional activity of DNA regulatory sequences in the mouse GLP-2R 5′-flanking region. The findings demonstrate that a ^{˜}1.5 kb fragment of the mouse GLP-2 receptor gene containing 5′-flanking and 5′-untranslated sequences directs transgene expression specifically to the GI tract and brain, in agreement with the restricted pattern of tissue-specific expression demonstrated for the endogenous GLP-2 receptor (Munroe et al., (1999) Proc. Natl. Acad. Sci. USA 96, 1569-1573; and Yusta et al., (2000) Gastroenterology 119(3), 744-755.)

The identification of potential Sp1 binding sites in the proximal GLP-2R promoter is intriguing in light of studies suggesting the functional importance of Sp1 binding sites for basal GLP-1 receptor transcription in transfection studies in vitro (Wildhage et al., (1999) Endocrinology 140(2), 624-31). Furthermore, several studies have demonstrated an important role for both GATA factors and caudal proteins (FIG. 7b) in the regulation of both intestine- and enteroendocrine-specific gene transcription. Traber et al., (1996) Annual Rev. Physiol. 58, 275-97; Boylan et al., (1997) J. Biol. Chem. 272(28), 17438-43; Dusing et al., (2000) Am. J. Physiol. 279(5), G1080-93; and Jin et al., (1996) Mol. Cell. Biol. 16, 19-28.

The regional and tissue-specific localization of GLP-2R promoter-LacZ expression was highly correlated with the expression of the endogenous murine GLP-2R, with the exception of the pituitary gland and lung. It was found however, that the abundance of cells expressing the endogenous GLP-2R appeared comparatively greater than the number of cells expressing the GLP-2R promoter-LacZ transgene in regions such as the hippocampus and cerebellum. These findings imply that additional DNA regulatory sequences not present in the 1.5 kb GLP-2R 5′-flanking region are required to correctly specify transgene expression in all cells and tissues expressing the endogenous GLP-2R receptor. Furthermore, the interpretation of the localization data may be further complicated in that unlike the endogenous GLP-2R, the nuclear LacZ reporter protein would not be transported to sites distal from the neural nuclei that transcribed the GLP-2R promoter in vivo.

What is claimed is:

1. A recombinant DNA construct, comprising a promoter region of a GLP-2 receptor gene and, linked for expression therewith, a heterologous gene of interest.

2. A recombinant DNA construct according to claim 1, wherein the promoter region is selected from the promoter region of the mouse GLP-2 receptor gene, the promoter region of a homolog of the mouse GLP-2 receptor gene, or a variant of such promoter regions that incorporates a truncation, insertion, deletion or addition and retains the function of a GLP-2 receptor gene promoter region.

3. A recombinant DNA construct according to claim 1, wherein the hetereologous gene of interest is a reporter gene.

4. A recombinant DNA construct according to claim 1, wherein the heterologous gene of interest encodes a therapeutic protein.

5. A cell incorporating a recombinant DNA construct as defined in claim 1.

6. A transgenic, non-human animal incorporating the recombinant DNA construct according to claim 1.

7. A method for screening compounds to identify regulators of GLP-2 receptor expression, the method comprising the steps of obtaining a reporter construct in which a reporter gene is linked for expression to a promoter region of a GLP-2 receptor gene, and determining the effect of a candidate regulator on the expression of said reporter gene.

8. A method for screening cells to identify hosts that will support expression from a GLP-2 receptor promoter region, comprising the step of culturing a selected host cell transfected with a recombinant DNA expression construct as defined in claim 3, measuring reporter gene expression level, and identifying such hosts as those in which elevated reporter gene expression levels are measured.
